(19) United States
(12) Patent Application Publication
(10) Pub. No.: US 2016/0304617 A1
(43) Pub. Date: Oct. 20, 2016

(54) 5T4-TARGETED IMMUNOFUSION MOLECULE AND METHODS

(71) Applicant: ASANA BIOSCIENCES, LLC, Bridgewater, NJ (US)

(72) Inventors: Nitin Damle, Berwyn, PA (US); Aranapakam Venkatesan, Chadds Ford, PA (US); Seetha Krishnan, Bangalore (IN); Priyaranjan Pattanaik, Bangalore (IN); Saurabh Joshi, Bangalore (IN)

(73) Assignee: Asana Biosciences, LLC, Bridgewater, NJ (US)

(21) Appl. No.: 14/899,470

(22) PCT Filed: Jun. 17, 2014
(86) PCT No.: PCT/US2014/042782

§ 371 Date: Dec. 17, 2015

Related U.S. Application Data
(60) Provisional application No. 61/835,858, filed on Jun. 17, 2013.

(51) Int. Cl. C07K 16/30; C12N 9/22

(52) U.S. Cl. CPC C07K 16/30; C07K 16/3023; C07K 16/3069; C12N 9/22; C12Y 301/00; C07K 2319/33; C07K 2317/622; C07K 2317/73; C07K 2317/94; A61K 2039/505

(57) ABSTRACT

Immunofusion molecules useful for 5T4-targeted therapy. The immunofusion molecules include the 5T4 anti-gen-binding portion of an anti-5T4 antibody engineered into a single chain form and fused to a cytotoxic payload, such as, human pancreatic RNase (“HPRN”). The RNase portion of the single immunofusion peptide may be fused to a polyglutamic acid (polyE) tail. A pharmaceutical composition includes an immunofusion molecule including a 5T4 antigen-binding portion and HPRN and methods of administering the composition to an animal in need.

### CROSS-REFERENCE TO RELATED APPLICATION

The present application claims the benefit of the filing date of U.S. Provisional Application No. 61/835,858, filed Jun. 17, 2013, entitled 5T4-TARGETED IMMUNOFUSION MOLECULE AND METHODS, the disclosure of which is hereby incorporated herein by reference.

### TECHNICAL FIELD

This disclosure relates to a 5T4-targeted immunofusion molecule comprising a 5T4-antigen-binding portion and a cytotoxic portion. The disclosure also relates to methods of synthesizing and using an immunofusion molecule for treatment of 5T4 antigen-related diseases.

### BACKGROUND

In some diseases, such as cancer, affected cells may exhibit altered expression of one or more surface antigens. In some circumstances, this cellular derangement may lead to a significant change in the level of expression of a certain antigen in a diseased cell compared to a healthy cell. Thus, a disease may have associated with it a specific antigen expression profile which may be crucial in the immune recognition, elimination, and control of the disease. Antigens that are particularly associated with disease can serve as an identifying marker of diseased cells and are valuable in the development of targeted therapies.

One example of an antigen associated with cancer is the human 5T4 antigen, also known as 5T4 oncofetal antigen. The human 5T4 antigen is a 72kDa type I transmembrane glycoprotein expressed in embryonic tissues, such as placenta, and in various types of solid tumors and carcinomas, including prostate cancer, gastric cancer, and colorectal cancer. See, e.g., U.S. Pat. No. 7,074,909 or U.S. Pat. No. 7,514,546. However, the 5T4 antigen is either expressed at low levels or not expressed in most healthy adult epithelial tissues. See Woods et al., Biochem. J. (2002) 366, 353-365.

The expression or overexpression of the 5T4 antigen in various tumor types, particularly in ovarian, gastric and colorectal tumors, is associated with poorer clinical outcomes. Id. Additionally, overexpression is associated with changes in cell morphology and motility that are consistent with tumor invasion. Thus, it is believed that the 5T4 antigen plays a role in the progression or malignancy of some solid tumors. Id. Due to the effects of 5T4 antigen expression on the characteristics of cells and its association with poor clinical outcome, the 5T4 antigen has been of interest for further study and characterization.

The association of certain antigens with cancer or other diseases gives rise to potential for use of the antigens in creating immunofusion molecules. Immunofusion molecules are effective as antigen-specific cytotoxic or cytostatic agents and have been developed for use in the manufacture of pharmaceutical compositions. See, e.g., WO2007/122511. Immunofusion molecules are genetically engineered proteins comprising of an antigen-binding portion derived from an antibody fused to a biologically active protein payload. In therapeutic applications, the antigen-binding portion of an immunofusion molecule may be derived from an antibody selective for cell-surface antigens associated with cancer or other diseases. This design provides for targeted delivery of a biologically active payload to the diseased cells and reduces impact on healthy cells that do not express the antigen target.

One example of a biologically active molecule is a ribonuclease (“RNase”), which is known to be useful for incorporation in immunofusion molecules as the cytotoxic or cytostatic payload. See, e.g., U.S. Pat. No. 5,840,840; U.S. Pat. No. 5,955,073; U.S. Pat. No. 6,045,793; U.S. Pat. No. 6,653,104; and U.S. Pat. No. 6,869,604; US 2010/0015661. RNases act to degrade RNA, thereby disabling the translational machinery of cells leading to preferential death of dividing cells. For example, human pancreatic RNase (“HPRN”) is one RNase that when introduced inside the cells is believed to play a role in both inducing cell death and increasing the susceptibility of cells to traditional chemotherapeutics. See, e.g., Leland, P. A., et al. (2001) Endowing Human Pancreatic Ribonuclease with Toxicity for Cancer Cells, Journal of Biological Chemistry, 276(46): 43095-43102. However, studies have shown that the level of activity of an immunofusion molecule varies significantly depending on the RNase and targeting moiety to which it is bound. See US 2005/0249738. For example, ONCONASE® (a frog RNase, also known as ranpirnase) when conjugated to the LL2 antibody, which is an anti-CD22 antibody, is dramatically more effective than the same antibody conjugated to either HPRN or eosinophil-derived neurotoxin RNase. Id. Accordingly, there is a recognized degree of unpredictability associated with the use of RNases as cytotoxic moieties of immunofusion molecules.

### SUMMARY

We provide immunofusion molecules useful for 5T4-targeted cancer therapy. The immunofusion molecules preferably comprise the 5T4 antigen-binding portion of an anti-5T4 antibody engineered into a single chain form and fused to a biologically active payload, such as human pancreatic RNase (“HPRN”). In its broadest aspect, the present invention is directed to immunofusion molecules comprising a 5T4 antigen-binding portion and a cytotoxic payload (e.g., RNase) portion in a single peptide chain. In some examples, an RNase portion of the single immunofusion peptide may be fused to a polyglutamic acid (polyE) tail to impart a negative charge to the fusion protein.

We further provide pharmaceutical compositions comprising an immunofusion molecule that includes a 5T4 antigen-binding portion and HPRN.

Additionally, we provide methods of treating diseases or disorders involving expression of the 5T4 antigen comprising administering to an animal in need of such treatment a therapeutically effective amount of a pharmaceutical composition comprising an immunofusion molecule comprising a 5T4 antigen-binding portion and an RNase portion in a single peptide chain.

### BRIEF DESCRIPTION OF THE DRAWINGS

FIG. 1 is a schematic diagram of an exemplary immunofusion molecule comprising the antigen-binding portion of an anti-5T4 antibody engineered into a single chain form and fused to HPRN.

FIG. 2 is a schematic diagram of an exemplary polypeptide sequence of an immunofusion molecule comprising the antigen-binding portion of an anti-5T4 antibody engineered into a single chain form and fused to HPRN. Residues represented by an asterisk (*) rather than a single-letter amino acid abbreviation are residues which may optionally be mutated or inserted into the polypeptide sequence.

FIG. 3 is a schematic representation of an exemplary anti-5T4 scFv-Fc construct design.

FIG. 4 is a schematic representation of an exemplary anti-5T4 scFv-Fc-HPRN construct design.

FIG. 5 is a schematic representation of an exemplary anti-5T4 scFv-Fc-HPRN-PolyE construct design.

FIG. 6 is a graph showing concentration dependent binding of HPRN immunofusion proteins to 5T4-overexpressing MDA-MB-231 breast carcinoma cells.

FIG. 7A is a histogram showing flow cytometric analysis of 5T4 expression in a MDAMB361 breast carcinoma cell line using anti-5T4-hu-scFv-Fc (triangle) and an unrelated (control) Fc fusion protein (circle) as probes.

FIG. 7B is a histogram showing flow cytometric analysis of 5T4 expression in a PC3 prostate carcinoma cell line using anti-5T4-hu-scFv-Fc (triangle) and an unrelated (control) Fc fusion protein (circle) as probes.

FIG. 7C is a histogram showing flow cytometric analysis of 5T4 expression in a PA1 endometrial carcinoma cell line using anti-5T4-hu-scFv-Fc (triangle) and an unrelated (control) Fc fusion protein (circle) as probes.

FIG. 7D is a histogram showing flow cytometric analysis of 5T4 expression in a A431 cervical carcinoma cell line using anti-5T4-hu-scFv-Fc (triangle) and an unrelated (control) Fc fusion protein (circle) as probes.

FIG. 7E is a histogram showing flow cytometric analysis of 5T4 expression in a SKOV3 ovarian carcinoma cell line using anti-5T4-hu-scFv-Fc (triangle) and an unrelated (control) Fc fusion protein (circle) as probes.

FIG. 7F is a histogram showing flow cytometric analysis of 5T4 expression in a HT29 colon carcinoma cell line using anti-5T4-hu-scFv-Fc (triangle) and an unrelated (control) Fc fusion protein (circle) as probes.

FIG. 7G is a histogram showing flow cytometric analysis of 5T4 expression in a DLD1 colon carcinoma cell line using anti-5T4-hu-scFv-Fc (triangle) and an unrelated (control) Fc fusion protein (circle) as probes.

FIG. 7H is a histogram showing flow cytometric analysis of 5T4 expression in a BxPC3 pancreatic carcinoma cell line using anti-5T4-hu-scFv-Fc (triangle) and an unrelated (control) Fc fusion protein (circle) as probes.

FIG. 7I is a histogram showing flow cytometric analysis of 5T4 expression in a LnCaP prostate carcinoma cell line using anti-5T4-hu-scFv-Fc (triangle) and an unrelated (control) Fc fusion protein (circle) as probes.

FIG. 7J is a histogram showing flow cytometric analysis of 5T4 expression in a human 5T4-transfected Rec-MDA-MB-231-5T4 breast carcinoma cell line using anti-5T4-hu-scFv-Fc (triangle) and an unrelated (control) Fc fusion protein (circle) as probes.

FIG. 7K is a histogram showing flow cytometric analysis of 5T4 expression in a MBA-MB-231 cancer cell line using anti-5T4-hu-scFv-Fc (triangle) and an unrelated (control) Fc fusion protein (circle) as probes.

FIG. 8A is graph of the dose response of cytotoxicity of anti-5T4 hu-scFv-Fc-HPRN (circles) against Recombinant MDA-MB-231-5T4 cells compared to a nonbinding scFv-Fc-HPRN (triangle).

FIG. 8B is graph of the dose response of cytotoxicity of anti-5T4 hu-scFv-Fc-HPRN (circles) against PC3 cells compared to a nonbinding scFv-Fc-HPRN (triangle).

FIG. 8C is graph of the dose response of cytotoxicity of anti-5T4 hu-scFv-Fc-HPRN (circles) against DLD1 cells compared to a nonbinding scFv-Fc-HPRN (square).

FIG. 8D is graph of the dose response of cytotoxicity of anti-5T4 hu-scFv-Fc-HPRN (circles) against PA1 cells.

FIG. 8E is graph of the dose response of cytotoxicity of anti-5T4 hu-scFv-Fc-HPRN (circles) against SKOV3 cells.

FIG. 8F is graph of the dose response of cytotoxicity of anti-5T4 hu-scFv-Fc-HPRN (circles) against A431 cells.

FIG. 9A is a schematic representation of an exemplary polynucleotide encoding a 5T4 scFv-Fc-CatB-HPRN-CatB-PolyE immunofusion molecule and optimized for expression in chinese hamster ovary cells.

FIG. 9B is a continuation of the schematic representation of an exemplary polynucleotide shown in FIG. 9A.

FIG. 9C is a continuation of the schematic representation of an exemplary polynucleotide shown in FIG. 9B.

### DETAILED DESCRIPTION

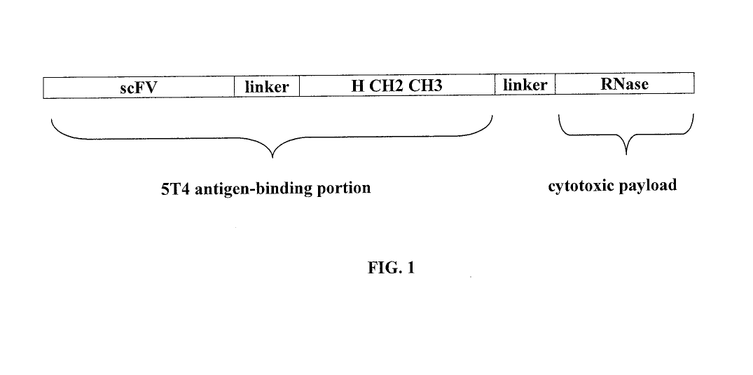

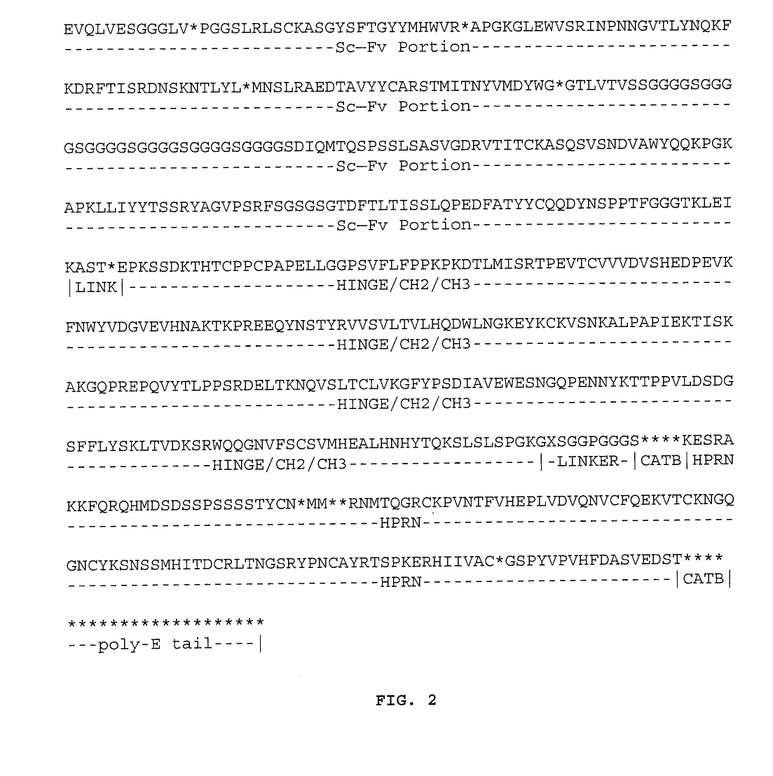

We provide 5T4-targeted immunofusion molecule derived from an anti-5T4 antibody that is specific for the 5T4 antigen. As used herein, “immunofusion molecule” refers to a protein or polypeptide generated by a genetic fusion two proteins such as by expressing a polypeptide encoded by a polynucleotide sequence encoding a portion of two or more genes. In preferred examples, an immunofusion molecule may comprise a “5T4 antigen-binding portion” and an “RNase portion” with its N-terminus linked to the C-terminus of the 5T4 antigen-binding portion, generally by a linker peptide. This arrangement is exemplified by the single chain fusion protein shown schematically in FIGS. 1 and 2 and described in detail below.

### Antigen-binding Portion

As used herein, the terms “5T4 antigen-binding portion” refers to a polypeptide sequence capable of selectively binding to the 5T4 antigen. In exemplary immunofusion molecules, the 5T4 antigen-binding portion generally comprises a single chain scFv-Fc form engineered from an anti-5T4 antibody. A single-chain variable fragment (scFvFc) is a fusion protein of the variable regions of the heavy (VH) and light chains (VL) of an immunoglobulin, connected with a linker peptide, and further connected to an Fc region comprising a hinge region and CH2 and CH3 regions of an IgG. Within such a scFvFc molecule, the scFv portion may be C-terminally linked to the N-terminus of the IgG Fc section by a linker peptide.

Generally, single chain antibody fragments have the same monomeric binding affinity as the Fab′ fragment of the parental monoclonal antibody, but can be conveniently expressed in a variety of hosts, including bacteria, yeast, and plants. See Worn et al., J. Mol. Biol. (2001) 305, 989-1010. Various techniques for engineering the scFvFc form of an antibody are known. See U.S. Pat. No. 7,189,393, Borras et al., J Biol Chem. 2010 Mar 19;285(12):9054-66; see also, Sambrook et al. (1989, Molecular Cloning: A Laboratory Manual, 2nd ed., Cold Spring Harbor Laboratory Press, Cold Spring Harbor, N.Y.). Briefly, mRNA encoding the variable regions of the heavy chain (VH) and light chain (VL) genes may be isolated and amplified using reverse-transcriptase polymerase chain reaction (RT-PCR). The amplified sequences for the heavy and light chains may be connected to one another and to a suitable hinge portion and constant domains CH2 and CH3 of human IgG1 to form a sequence encoding a single polypeptide chain. Additionally, the nucleic acids encoding complementarity determining regions (CDRs) may be grafted onto acceptor frameworks or scaffolds with suitable biophysical properties. Id.

At least a portion of the 5T4 antigen-binding portion of our immunofusion molecules may originate from a murine source. For example, one may obtain an immunofusion molecule by expressing a polynucleotide engineered to encode at least a murine anti-5T4 scFv region having the polypeptide sequence according to SEQ ID NO:4. Additionally, at least a portion of the 5T4-antigen binding portion may be generated to be chimeric or humanized according to well known methods. See Borras, supra. Thus, one may obtain an immunofusion molecule having a 5T4-antigen binding portion as a humanized scFv portion by expressing a polynucleotide engineered to encode at least the polypeptide sequence according to SEQ ID NO:5.

In some examples, the Fv portion of the 5T4 antigen-binding portion may be engineered by molecular techniques to comprise one or more amino acid substitutions in the VH region. Preferably, the polynucleotide encoding the polypeptide of the scFv portion is modified to encode a sequence having one or more glutamine residues of the VH region substituted with glutamic acid residues. For example, glutamine residues at positions 13, 39, 82, and/or 112 of SEQ ID NO:5, may be substituted with glutamic acid residues to obtain SEQ ID NO:96. Substitution of glutamine residues with glutamic acid residues reduces the positive charge of the immunofusion molecule. While not wishing to be bound by theory, it is believed that a reduction in a positive charge contributes to increased solubility and improved expression, pharmacokinetics and possibly intratumoral penetration of the immunofusion molecules.

The Fc portion of the 5T4 antigen binding portion preferably comprises a polypeptide sequence engineered from the human hinge, CH2 and CH3 domains of human IgG1. For, example, it is possible to engineer a polynucleotide to encode at least an Fc portion having the polypeptide sequence according to SEQ ID NO:8.

A polypeptide linker, such as one having the polypeptide sequence ASTC (SEQ ID NO:6) or ASTX (SEQ ID NO:7) (where “X” refers to any amino acid or a direct peptide bond between the adjacent amino acids), may fuse the C-terminus of scFv portion to the N-terminus of the Fc portion of the 5T4 antigen-binding portion. Thus, it is possible to engineer a polynucleotide to encode at least a linker having the polypeptide sequence according to SEQ ID NOs:6 or 7. While either SEQ ID NOs:6 or 7 may be used as a linker, an immunofusion molecule having a peptide linker according to SEQ ID NO:6 benefits from the potential and opportunity for site-specific conjugation due to the presence of the cysteine residue.

A polynucleotide encoding a peptide wherein the single chain Fv and Fc regions are linked together may encode at least a chimeric 5T4 antigen-binding portion of an immunofusion molecule having the polypeptide sequence according to SEQ ID NO:21 or may encode a humanized 5T4 antigen-binding portion having the polypeptide sequence according to SEQ ID NOs:22 or 23. A humanized 5T4 antigen-binding portion may also have one or more substitutions of the glutamine residues at positions 13, 39, 82, and/or 112 and have a polypeptide sequence according to SEQ ID NO:97 or 98.

Additionally, a nucleotide sequence encoding an N-terminal peptide signal sequence for murine IgVH according to SEQ ID NO:2 may be included in the polynucleotide encoding the scFv-Fc 5T4 antigen-binding portion of an immunofusion molecule. However, the signal peptide is post-translationally cleaved and is not a part of the functional immunofusion molecule.

### Linker

A polynucleotide encoding an immunofusion molecule may be engineered to encode at least one polypeptide linker sequence linking a 5T4 antigen-binding portion to an RNase portion. A polypeptide linker preferably fuses the C-terminus of the scFv-Fc antigen-binding portion to the N-terminus of the RNase portion.

Linker peptides are known and may generally comprise between one and twenty amino acids. In exemplary immunofusion molecules, the polypeptide linker may be a polypeptide sequence according to SEQ ID NO:9, SEQ ID NO:10, SEQ ID NO:11 or SEQ ID NO:12. In these polypeptide linkers, the second amino acid residue may be either cysteine or serine.

Additionally, the linker may be engineered to have a four-amino-acid CathepsinB (“CatB”) substrate sequence on the C-terminal end such as that shown in SEQ ID NOs:11 and 12. As shown in SEQ ID NOs:11 and 12, a CatB sequence is a C-terminal polypeptide sequence which can be represented as glycine-leucine-phenylalanine-arginine (GLFR). Alternatively, a CatB sequence may have an amino acid sequence according to any one of the following: GLVR (SEQ ID NO:104), GLFRFFG (SEQ ID NO:105), GLVRAFG (SEQ ID NO:106), GLFRAFG (SEQ ID NO:107) or LLVRFFG (SEQ ID NO:108).

A CatB sequence is a target for cleavage by Cathepsin B after the immunofusion molecule is internalized via 5T4-mediated internalization. A CatB sequence may be introduced into the immunofusion molecule N-terminally to the RNase portion or both N-terminal and C-terminal to the RNase portion. Thus, the immunofusion molecule may be engineered to allow cleavage of the RNase portion from the 5T4 antigen-binding portion after the immunofusion molecule is internalized by a cell. Additionally, the immunofusion molecule may be engineered to allow cleavage of the RNase portion from a C-terminal peptide after the immunofusion molecule is internalized by a cell.

### RNase

In selected, illustrative examples, the immunofusion molecules may be obtained by expressing at least a polynucleotide encoding an RNase. Preferably, the N-terminus of the RNase portion may be fused to the C-terminus of a linker peptide fused to the 5T4 antigen-binding portion. A preferred RNase is HPRN, which is a protein having 128 amino acid residues according to the polypeptide sequence of SEQ ID NO:13.

Several HPRN muteins have been identified (see Leland et al., supra) and may be used in the immunofusion molecules of this disclosure such as by utilizing known techniques for engineering site specific mutations. For example, Q28L and E111G have been identified as increasing the cytotoxicity of HPRN. The polypeptide sequences of these HPRN muteins are shown in SEQ ID NOs:14 and 16, respectively. As shown in SEQ ID NO:14, the glutamine residue (Q) at position 28 of SEQ ID NO:13 is replaced with a glutamic acid residue (E). As shown in SEQ ID NO:16, the glutamic acid residue (E) at position 111 of SEQ ID NO:13 is replaced with a glycine residue (G). Additionally or alternatively, the polypeptide sequence of the HPRN protein may be modified by a R31C and R32C double mutation, as shown in SEQ ID NO:15. Thus, the arginine residues (R) at positions 31 and 32 of SEQ ID NO:13 are replaced with cysteine residues (C).

The RNase portion of the immunofusion molecule may be engineered to include a combination of one or more of these mutations. For example, the RNase portion of the immunofusion molecule may be encoded by a polynucleotide encoding the polypeptide sequence according to any one of SEQ ID NO:13 to SEQ ID NO:20. For example, the RNase portion of an immunofusion molecule may be any one of the following:

1. wildtype HPRN (SEQ ID NO:13)

2. Q28L (SEQ ID NO:14)

3. E111G (SEQ ID NO:16)

4. R31C/R32C (SEQ ID NO:15)

5. Q28L/R31C/R32C (SEQ ID NO:17)

6. R31C/R32C/E111G, (SEQ ID NO:18)

7. Q28L/E111G (SEQ ID NO:19), or

8. Q28L/R31C/R32C/E111G (SEQ ID NO:20).

The immunofusion molecules may dimerize upon expression in a cell. Alternatively, the immunofusion molecules may form a tetrameric structure. While not wishing to be bound to theory, it is believed that the R31C and R32C double mutation contributes to the formation of dimeric or tetrameric complexes upon expression of the immunofusion molecules in a cell.

In some examples, the RNase portion of the immunofusion molecule may be fused with an additional C-terminal tail of poly-glutamic acid (“polyE”), which imparts a negative charge to the fusion protein. The number of glutamic acid residues may be 5 or more and 150 or less. Preferably, 10 or more and 100 or less, or 15 or more and 50 or less. A polyglutamated immunofusion protein may have the following organization:

scFv-Fc-CatB-RNase-CatB-polyE

Often, proteins with clustered positively charged molecules such as RNases are retained in the heparin sulfate proteoglycan (HSPG) that surrounds vascular endothelial cells. In some instances, chondroitin sulfate proteoglycan can also serve the same function. These sulfated (negatively charged) matrices help syphon out proteins with clustered positive charge from circulation in the blood. Accordingly, systemic delivery of positively charged molecules such as RNases may result in at least partial sequestration of the molecules and unfavorable pharmacokinetics.

Introduction of a negative charge such as with a polyE tail, reduces or avoids electrostatic retention in the blood vessels of perfused organs such as liver, spleen, and bone marrow and thereby increases the presence of polyE-tailed macromolecule therapeutics in circulation for intratumoral accumulation. Thus, a polyglutamated scFv-Fc-RNase immunofusion molecule is believed to have improved pharmacokinetic profile than a scFv-Fc-RNase immunofusion molecule without a polyE tail.

Exemplary immunofusion molecules have the polypeptide sequences according to any one of SEQ ID NOs:32 to 95, which incorporate the variations in polypeptide sequences of the 5T4 antigen binding portion, linkers and the RNase protein discussed above. Other exemplary immonofusion molecules have the polypeptide sequences designated as SEQ ID NOs:24 to 31. These sequences incorporate the variations in polypeptide sequences of the 5T4 antigen binding portions, linkers and optionally CathepsinB (CatB) substance sequences.

In addition to the examples described above, the immunofusion molecules may comprise one or more amino acid substitutions in the polypeptide sequences of the 5T4-antigen binding portion and/or the RNase portion. In some examples, amino acid substitutions are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

In other examples, there may be one or more amino acid insertions or deletions in the polypeptide sequences of the 5T4 antigen-binding portion and/or the RNase portion. “Insertions” or “deletions” may be about 1 to 5 amino acids, or more. The variation allowed may be experimentally determined by systematically making insertions, deletions, or substitutions of amino acids in a polypeptide molecule using recombinant DNA techniques and assaying the resulting recombinant variants for activity. This does not require more than routine experiments for the skilled artisan.

In some examples, one or more amino acids in the polypeptide sequence encoding the 5T4 antigen-binding portion and/or the RNase portion may be replaced with a peptidomimetic. Peptidomimetics are compounds containing non-peptidic structural elements that are capable of mimicking or antagonizing the biological action(s) of a natural peptide. In general, peptidomimetics can be classified into two categories. The first includes compounds with non-peptide-like structures, often scaffolds onto which pharmacophoric groups have been attached. Thus, they are low molecular-weight compounds and bear no structural resemblance to the native peptides, resulting in an increased stability towards proteolytic enzymes. The second main class of peptidomimetics includes compounds of a modular construction comparable to that of peptides. These compounds can be obtained by modification of either the peptide side chains or the peptide backbone. Peptidomimetics of the latter category can be considered to be derived of peptides by replacement of the amide bond with other moieties. As a result, the compounds are expected to be less sensitive to degradation by proteases. Modification of the amide bond also influences other characteristics such as lipophilicity, hydrogen bonding capacity and conformational flexibility, which in favorable cases may result in an overall improved pharmacological and/or pharmaceutical profile of the compound.

Suitable peptidomimetics for use in the immunofusion molecules are amide bond surrogates such as the oligo-β-peptides (Juaristi, E. Enantioselective Synthesis of b-Amino Acids; Wiley-VCH: New York, 1996), vinylogous peptides (Hagihari, M. et al., J. Am. Chem. Soc. 1992, 114, 10672-10674), peptoids (Simon, R. J. et al., Proc. Natl. Acad. Sci. USA 1992, 89, 9367-9371; Zuckermann, R. N. et al., J. Med. Chem. 1994, 37, 2678-2685; Kruijtzer, J. A. W. & Liskamp, R. M. J. Tetrahedron Lett. 1995, 36, 6969-6972); Kruijtzer, J. A. W. Thesis; Utrecht University, 1996; Kruijtzer, J. A. W. et al., Chem. Eur. J. 1998, 4, 1570-1580), oligosulfones (Sommerfield, T. & Seebach, D. Angew. Chem., Int. Ed. Eng. 1995, 34, 553-554), phosphodiesters (Lin, P. S.; Ganesan, A. Bioorg. Med. Chem. Lett. 1998, 8, 511-514), oligosulfonamides (Moree, W. J. et al., Tetrahedron Lett. 1991, 32, 409-412; Moree, W. J. et al., Tetrahedron Lett. 1992, 33, 6389-6392; Moree, W. J. et al., Tetrahedron 1993, 49, 1133-1150; Moree, W. J. Thesis; Leiden University, 1994; Moree, W. J. et al., J. Org. Chem. 1995, 60, 5157-5169; de Bont, D. B. A. et al., Bioorg. Med. Chem. Lett. 1996, 6, 3035-3040; de Bont, D. B. A. et al., Bioorg. Med. Chem. 1996, 4, 667-672; Lowik, D. W. P. M. Thesis; Utrecht University, 1998), peptoid sulfonamides (van Ameijde, J. & Liskamp, R. M. J. Tetrahedron Lett. 2000, 41, 1103-1106), vinylogous sulfonamides (Gennari, C. et al., Eur. J. Org. Chem. 1998, 2437-2449), azatides (or hydrazinopeptides) (Han, H. & Janda, K. D. J. Am. Chem. Soc. 1996, 118, 2539-2544), oligocarbamates (Paikoff, S. J. et al., Tetrahedron Lett. 1996, 37, 5653-5656; Cho, C. Y. et al., Science 1993, 261, 1303-1305), ureapeptoids (Kruijtzer, J. A. W. et al., Tetrahedron Lett. 1997, 38, 5335-5338; Wilson, M. E. & Nowick, J. S. Tetrahedron Lett. 1998, 39, 6613-6616) and oligopyrrolinones (Smith III, A. B. et al., J. Am. Chem. Soc. 1992, 114, 10672-10674). However, it is understood that other peptidomimetics may be used.

Preferably, any amino acid substitution, insertion, or deletion or use of a peptidomimetic does not substantially reduce the affinity or specificity of the 5T4 antigen-binding portion or the cytotoxicity of the RNase portion. An immunofusion molecule having an amino acid substitution, insertion, or deletion or a peptidomimetic in the 5T4 antigen-binding portion preferably retains greater than 75%, preferably greater than 80%, preferably greater than 85%, preferably greater than 90%, or preferably greater than 95% of affinity or specificity for binding the 5T4 antigen compared to the immunofusion molecule with an unmodified 5T4-antigen binding portion. Additionally, an immunofusion molecule having an amino acid substitution, insertion, or deletion or a peptidomimetic in the RNase portion preferably retains greater than 75%, preferably greater than 80%, preferably greater than 85%, preferably greater than 90%, or preferably greater than 95% of cytotoxic activity compared to the immunofusion molecule with an unmodified RNase portion.

We further provide an expression system for expressing a nucleic acid sequence coding for an anti-5T4 immunofusion molecule. This expression system preferably comprises one or more regulatory sequences. An expression system can comprise a transcriptional unit comprising an assembly of (1) a “control region” or genetic element or elements having a regulatory role in gene expression, for example, promoters or enhancers, (2) a structural or coding sequence which is transcribed into mRNA and translated into protein, and (3) appropriate transcription initiation and termination sequences. Structural units intended for use in eukaryotic expression systems may include a leader or signal sequence enabling extracellular secretion of translated protein by a host cell. One skilled in the art would further understand that the polynucleotide sequence encoding an immunofusion molecule may be altered for expression of the mature protein in a chosen expression system or organism.

Preferably, an expression system includes polynucleotide sequences that code for a selection marker (e.g., resistance to antibiotics, fungicides, or herbicides), a multiple cloning site containing the sites of restriction enzymes suitable for the insertion of DNA, and the cell/host system is preferably an inducible system Cohn et al., 1997, Eur. J. Biochem., 249, 473-480; Patry et al. (1994, FEBS Lett., 349(1): 23-8). An expression vector may be a plasmid.

We further provide host cells which have been transformed to contain polynucleotides encoding an immunofusion molecule. Preferably, a host cell can be a higher eukaryotic host cell such as a mammalian or plant cell, a lower eukaryotic host cell such as a yeast cell, or can be an insect cell, or the host cell can be a prokaryotic cell such as a bacterial cell such as E. coli. Mammalian cells may be a CHO, COS, HeLa, 293T, HEH or BHK cells. The term “transformation” means introducing DNA into a suitable host cell so that the DNA is replicable, either as an extrachromosomal element, or by chromosomal integration according to any suitable method such as the methods described by Sambrook et al. (1989, Molecular Cloning: A Laboratory Manual, 2nd ed., Cold Spring Harbor Laboratory Press, Cold Spring Harbor, N.Y.). Additionally, host cells may be genetically engineered to express the peptides encoded by the introduced polynucleotides, wherein the polynucleotides are in operative association with a regulatory sequence heterologous to the host cell which drives expression of the polynucleotides in the cell.

A preferred host cell and expression system utilizes members of the monocotyledonous family Lemnaceae, commonly referred to as “duckweed.” Duckweed plant or duckweed nodule cultures can be efficiently transformed with an expression cassette containing a polynucleotide sequence encoding 5T4-targeted immunofusion molecules by any one of a number of methods including Agrobacterium-mediated gene transfer, ballistic bombardment, or electroporation. Stable duckweed transformants can be isolated by transforming the duckweed cells with both the nucleotide sequence of interest and a gene that confers resistance to a selection agent, followed by culturing the transformed cells in a medium containing the selection agent. See U.S. Pat. Nos. 7,632,983, 6,815,184 and US Patent Pub. 2010/0043099.

A method of producing an immunofusion molecule in a duckweed plant culture or a duckweed nodule culture may comprise the steps of: (a) culturing within a duckweed culture medium a duckweed plant culture or a duckweed nodule culture, wherein the duckweed plant culture or the duckweed nodule culture is stably transformed to express one or more immunofusion peptide sequences; and (b) collecting the immunofusion peptide from the duckweed culture medium. In some examples, immunofusion molecules are expressed from a nucleotide sequence comprising a coding sequence for the immunofusion molecule and an operably linked coding sequence for a signal peptide that directs secretion of the immunofusion peptide into the culture medium. The stably transformed duckweed plant culture or duckweed nodule culture may be at least one selected from the group consisting of Lemna minor, Lemna miniscula, Lemna aequinoctialis, and Lemna gibba.

In some examples of a method of producing immunofusion molecules in duckweed culture, the nucleotide sequence encoding a polypeptide of a 5T4-targeted immunofusion molecule may have one or more attributes selected from the group consisting of: (a) duckweed-preferred codons in the coding sequence for said polypeptide; (b) duckweed-preferred codons in the coding sequence for a signal peptide; (c) a translation initiation codon that is flanked by a plant-preferred translation initiation context nucleotide sequence; (d) an operably linked nucleotide sequence comprising a plant intron that is inserted upstream of the coding sequence; and (e) an operably linked nucleotide sequence comprising the ribulose-bis-phosphate carboxylase small subunit 5B gene of Lemna gibba.

Stably transformed duckweed may be obtained by transformation with a nucleotide sequence of interest such as a nucleotide sequence encoding our immunofusion molecules, contained within an expression cassette. An expression cassette preferably comprises a transcriptional initiation region linked to the nucleic acid encoding the peptide sequence of an immunofusion molecule. Such an expression cassette may be provided with a plurality of restriction sites for insertion of the polynucleotide encoding an immunofusion molecule to be under the transcriptional regulation of the regulatory regions. In particular examples, nucleic acids to be transferred may be contained in two or more expression cassettes, each of which encodes at least one immunofusion molecule. For example, one expression cassette may comprise the polynucleotide encoding the 5T4-antigen binding portion and RNase portion of the immunofusion molecule whereas a second expression cassette comprises a gene encoding a protein that assists in the expression of the immunofusion molecules. One example of such a protein may be protein that inhibits enzymatic cleavage of the immunofusion molecules while they are present in the host cell. Alternatively, multiple expression cassettes may be provided.

For expression in duckweed and other expression systems, any suitable known promoter can be employed (including bacterial, yeast, fungal, insect, mammalian, plant promoters and the like). For example, plant promoters, including duckweed promoters, may be used. Exemplary promoters include, but are not limited to, the Cauliflower Mosaic Virus 35S promoter, the opine synthetase promoters (e.g., nos, mas, ocs, etc.), the ubiquitin promoter, the actin promoter, the ribulose bisphosphate (RubP) carboxylase small subunit promoter, and the alcohol dehydrogenase promoter. The duckweed RubP carboxylase small subunit promoter is known in the art (Silverthome et al. (1990) Plant Mol. Biol. 15:49). Other promoters from viruses that infect plants, preferably duckweed, are also suitable including, but not limited to, promoters isolated from Dasheen mosaic virus, Chlorella virus (e.g., the Chlorella virus adenine methyltransferase promoter; Mitra et al. (1994) Plant Mol. Biol. 26:85), tomato spotted wilt virus, tobacco rattle virus, tobacco necrosis virus, tobacco ring spot virus, tomato ring spot virus, cucumber mosaic virus, peanut stump virus, alfalfa mosaic virus, sugarcane baciliform badnavirus and the like.

Promoters can also be chosen to give a desired level of regulation. For example, in some instances, it may be advantageous to use a promoter that confers constitutive expression (e.g., the mannopine synthase promoter from Agrobacterium tumefaciens). Alternatively, in other situations, it may be advantageous to use promoters activated in response to specific environmental stimuli (e.g., heat shock gene promoters, drought-inducible gene promoters, pathogen-inducible gene promoters, wound-inducible gene promoters, and light/dark-inducible gene promoters) or plant growth regulators (e.g., promoters from genes induced by abscissic acid, auxins, cytokinins, and gibberellic acid). As a further alternative, promoters can be chosen that give tissue-specific expression (e.g., root, leaf, and floral-specific promoters).

In general, a transcriptional cassette may include in the 5′-3′ direction of transcription, a transcriptional and translational initiation region, a nucleotide sequence of interest, and a transcriptional and translational termination region functional in plants. Any suitable known termination sequence may be used. The termination region may be native with the transcriptional initiation region, may be native with the nucleotide sequence of interest, or may be derived from another source. Exemplary termination regions are available from the Ti-plasmid of A. tumefaciens, such as the octopine synthetase and nopaline synthetase termination regions. See also Guerineau et al. (1991) Mol. Gen. Genet. 262:141; Proudfoot (1991) Cell 64:671; Sanfacon et al. (1991) Genes Dev. 5:141; Mogen et al. (1990) Plant Cell 2:1261; Munroe et al. (1990) Gene 91:151; Ballas et al. (1989) Nucleic Acids Res. 17:7891; and Joshi et al. (1987) Nucleic Acids Res. 15:9627. Additional exemplary termination sequences are the pea RubP carboxylase small subunit termination sequence and the Cauliflower Mosaic Virus 35S termination sequence. Other suitable termination sequences will be apparent to those skilled in the art.

Generally, an expression cassette may comprise a selectable marker gene for the selection of transformed cells or tissues. Selectable marker genes include genes encoding antibiotic resistance such as those encoding neomycin phosphotransferase II (NEO) and hygromycin phosphotransferase (HPT), as well as genes conferring resistance to herbicidal compounds. Herbicide resistance genes generally code for a modified target protein insensitive to the herbicide or for an enzyme that degrades or detoxifies the herbicide in the plant before it can act. See DeBlock et al. (1987) EMBO J. 6:2513; DeBlock et al. (1989) Plant Physiol. 91:691; Fromm et al. (1990) BioTechnology 8:833; Gordon-Kamm et al. (1990) Plant Cell 2:603; and Frisch et al. (1995) Plant Mol. Biol. 27:405-9. For example, resistance to glyphosphate or sulfonylurea herbicides has been obtained using genes coding for the mutant target enzymes, 5-enolpyruvylshikimate-3-phosphate synthase (EPSPS) and acetolactate synthase (ALS). Resistance to glufosinate ammonium, boromoxynil, and 2,4-dichlorophenoxyacetate (2,4-D) have been obtained by using bacterial genes encoding phosphinothricin acetyltransferase, a nitrilase, or a 2,4-dichlorophenoxyacetate monooxygenase, which detoxify the respective herbicides.

The nucleotide sequence encoding immunofusion molecule may be modified to enhance its expression in duckweed or other host cells. As stated above, one such modification is the synthesis of the nucleotide sequence of interest using duckweed-preferred codons. Methods are available for synthesizing nucleotide sequences with plant-preferred codons. See, e.g., U.S. Pat. Nos. 5,380,831 and 5,436,391; Perlak et al. (1991) Proc. Natl. Acad. Sci. USA 15:3324; Iannacome et al. (1997) Plant Mol. Biol. 34:485; and Murray et al., (1989) Nucleic Acids. Res. 17:477, herein incorporated by reference. The preferred codons may be determined from the codons of highest frequency in the proteins expressed in duckweed. It is recognized that genes that have been modified for expression in duckweed and other monocots can be used in our methods. See, e.g., EP 0 359 472, EP 0 385 962, WO 91/16432; Perlak et al. (1991) Proc. Natl. Acad. Sci. USA 88:3324; Iannacome et al. (1997) Plant Mol. Biol. 34:485; and Murray et al. (1989) Nuc. Acids Res. 17:477, and the like, herein incorporated by reference. It is further recognized that all or any part of the nucleotide sequence may be modified or synthetic. In other words, fully modified or partially modified sequences may also be used. For example, 40%, 45%, 50%, 55%, 60%, 65%, 70%, 75%, 80%, 85%, 87%, 90%, 91%, 92%, 93%, 94%, 95%, 96%, 97%, 98%, 99%, 100% or any amount therebetween of the codons may be modified codons. In one example, between 90 and 96% of the codons are modified codons.

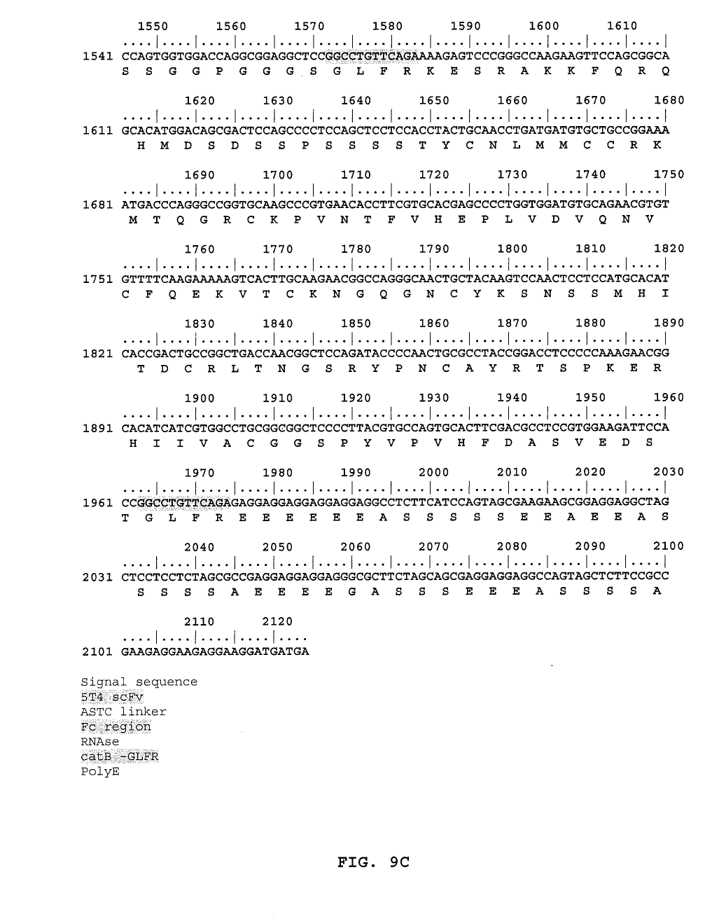

A suitable CHO modified polynucleotide sequence encoding an immunofusion molecules of this disclosure may be according to SEQ ID NO:109. FIGS. 9A to 9C show a schematic representation of a polynucleotide encoding an anti-5T4 scFv-Fc-HPRN-PolyE construct (SEQ ID NO:109). As shown, polynucleotides 1 to 62 encode a signal sequence, polynucleotides 63 to 833 (shown with gray background) encode a 5T4 ScFv, polynucleotides 834 to 840 encode a linker sequence, polynucleotides 841 to 1536 (shown with gray background) encode an Fc region, polynucleotides 1572 to 1583 (shown with gray background) encode a first CatB sequence, polynucleotides 1584 to 1960 encode an HPRN RNase, polynucleotides 1963 to 1974 (shown with gray background) encode a second CatB sequence, and polynucleotides 1975 to 2118 encode a PolyE tail. The encoded protein is shown in SEQ ID NO:113.

Codon modified sequences may be designed by any method known in the art, including the use of software programs, such as Vector NTI®. Suitable polynucleotides also include polynucleotides according to SEQ ID NOs:110 to 112.

Additionally, to facilitate secretion of immunofusion proteins from a cell, the nucleotide encoding the immunofusion peptide may also encode a “signal peptide” that interacts with a receptor protein on the membrane of the endoplasmic reticulum (ER) to direct the translocation of the polypeptide chain across the membrane and into the endoplasmic reticulum for secretion from the cell. This signal peptide is preferably cleaved from the precursor polypeptide to produce a “mature” polypeptide lacking the signal peptide. Thus, in one example, a biologically active polypeptide is expressed in duckweed from a nucleotide sequence operably linked with a nucleotide sequence encoding a signal peptide that directs secretion of the polypeptide into the culture medium. Plant signal peptides that target protein translocation to the endoplasmic reticulum (for secretion outside of the cell) are known in the art. See, for example, U.S. Pat. No. 6,020,169 to Lee et al. Any plant signal peptide can be used to target polypeptide expression to the ER. In some examples, the signal peptide is the Arabidopsis thaliana basic endochitinase signal peptide (amino acids 14-34 of NCBI Protein Accession No. BAA82823), the extension signal peptide (Stiefel et al. (1990) Plant Cell 2:785-793) or the rice alpha-amylase signal peptide (amino acids 1-31 of NCBI Protein Accession No. AAA33885). In another example, the signal peptide may correspond to the signal peptide of a secreted duckweed protein. Alternatively, a mammalian signal peptide can be used to target recombinant polypeptides expressed in genetically engineered host cells for secretion. An example of a signal peptide (e.g., ER localization signal) is KEDL (SEQ ID NO:3).

Stably transformed duckweed can be obtained by any known method such as the gene transfer methods disclosed in U.S. Pat. No. 6,040,498 to Stomp et al., herein incorporated by reference. These methods include gene transfer by ballistic bombardment with microprojectiles coated with a nucleic acid comprising the nucleotide sequence of interest, gene transfer by electroporation, and gene transfer mediated by Agrobacterium comprising a vector comprising the nucleotide sequence of interest. In one example, the stably transformed duckweed is obtained via any one of the Agrobacterium-mediated methods disclosed in U.S. Pat. No. 6,040,498 to Stomp et al. The Agrobacterium used may be Agrobacterium tumefaciens or Agrobacterium rhizogenes.

Stably transformed duckweed plants may also be obtained by chloroplast transformation. See, for example, U.S. provisional patent application No. 60/492,179, filed Aug. 1, 2003, entitled “Chloroplast transformation of duckweed.” Stably transformed duckweed lines may also be produced using plant virus expression vectors. See, for example, U.S. Pat. No. 6,632,980 and Koprowski and Yusibov (2001) Vaccine 19:2735-2741.

Methods of producing a substantially pure immunofusion protein may comprise growing a culture of the cells in a suitable culture medium, and purifying the protein from the culture. For example, the methods include a process of producing a polypeptide in which a host cell containing a suitable expression vector that includes a polynucleotide is cultured under conditions that allow expression of the encoded polypeptide. The polypeptide can be recovered from the culture, conveniently from the culture medium when the proteins are secreted from the host cells into subsequently enter the culture medium, and can be further purified. The resulting expressed protein may, for example, be purified from such culture (i.e., from culture medium or cell extracts) using known purification processes such as gel filtration and ion exchange chromatography.

We also provide methods of treating diseases or disorders involving the expression of the 5T4 antigen comprising administering to a patient or animal in need of such treatment a therapeutic composition comprising a therapeutically effective amount of an immunofusion molecule comprising a 5T4 antigen-binding portion and an RNase portion in a single peptide chain. Diseases associated with the expression of the 5T4 antigen include, but are not limited to, carcinoma and solid tumor cancers such as breast, bladder, cervical, colorectal, endometrial, gastric, head and neck, hepatic, lung, ovarian, pancreatic, renal and prostate carcinomas and others. Preferred examples include methods of treating bladder cancer and prostate cancer by administration of the immunofusion molecules of this disclosure to a patient suffering from bladder cancer or prostate cancer.

Administration of therapeutic compositions comprising such immunofusion proteins can be implemented, e.g., via the subcutaneous, intradermal, intraperitoneal or intravenous route, inhalation, intratumoral injection, intravesical instillation or any other suitable route.

Such a therapeutic composition may also contain (in addition to the ingredient and the carrier) diluents, fillers, salts, buffers, stabilizers, solubilizers and other well known materials. The term “pharmaceutically acceptable” means a non-toxic material that does not interfere with the effectiveness of the biological activity of the active ingredient(s). The characteristics of the carrier will depend on the route of administration. The therapeutic composition may further contain other agents which either enhance the activity or use in treatment. Such additional factors and/or agents may be included in the therapeutic composition to produce a synergistic effect or to minimize side-effects.

Pharmaceutical compositions are preferably sterile. Pharmaceutical compositions, in addition to at least one immunofusion molecule, preferably have at least one pharmaceutically acceptable carrier. Suitable pharmaceutically acceptable carriers include water (e.g., sterile water for injection); saline solutions such as physiological saline or phosphate buffered saline (PBS); polyethylene glycols, glycerine, propylene glycol, mannitol or other synthetic solvents; antibacterial agents such as benzyl alcohol or methyl parabens; antioxidants such as ascorbic acid or sodium bisulfite; buffers such as acetates, citrates or phosphates and agents for the adjustment of tonicity such as sodium chloride or dextrose; stabilizing or preservative agents, such as sodium bisulfite, sodium sulfite and ascorbic acid, citric acid and its salts, ethylenediaminetetraacetic acid, benzalkonium chloride, methyl- or propylparaben chlorobutanol; and combinations thereof.

Techniques for formulation and administration of our compounds may be found in “Remington's Pharmaceutical Sciences”, Mack Publishing Co., Easton, Pa. The compositions contain a therapeutically effective amount or dose of the respective ingredient. A therapeutically effective dose refers to that amount of the compound sufficient to result in amelioration of symptoms, e.g., treatment, healing or amelioration of such conditions. The dose will be dependent upon the properties of the immunofusion molecule employed, e.g., its activity and biological half-life, the concentration of the immunofusion molecule in the formulation, the site and rate of dosage, the clinical tolerance of the patient involved, the extent of cancer afflicting the patient and the like as is well within the skill of the physician. The physician can determine the actual dosage which will be most suitable for an individual patient and will vary with the age, weight and response of the particular patient. There can, of course, be individual instances where higher or lower dosage ranges are merited. The therapeutic compositions containing the immunofusion molecules may be administered in a therapeutically effective dose over either a single day or several days. Generally, in the case where a therapeutic composition comprising an immunofusion molecule is administered directly or systemically to a host, an infusion may be administered in the range of about 0.01-50 mg/kg, more usually from about 0.1-25 mg/kg body weight of the host.

Additionally, we provide methods of intravesical instillation of a therapeutic composition comprising an immunofusion molecule. Intravesical instillation comprises administration of a therapeutic composition comprising an immunofusion molecule directly into the bladder, generally through a urethral catheter. The theraeutic composition is generally held in the bladder for a “dwell time” before the bladder is drained or voided. This procedure allows the treatment of the urinary bladder wall directly with high concentrations of immunofusion molecules. Bladder instillation techniques are known. See, U.S. Pat. No. 7,025,753, U.S. Pat. No. 7,671,026, U.S. Pat. No. 6,630,515; and U.S. Pat. No. 4,871,542.

We further provide methods of combination therapy. In such methods, the immunofusion molecules may be administered to a patient in combination with chemotherapeutic agents. For example, our immunofusion molecules may be administered at the same time or in this the same infusion or instillation as one or more chemotherapeutic agents. Alternatively, our immunofusion molecules may be administered separately from chemotherapeutic agent or by separate administration techniques, but while the chemotherapeutics are active in the patient's body. Suitable chemotherapeutic agents for combination therapy may include, but are not limited to, at least one selected from the group consisting of cisplatin, carboplatin, cytarabine, gemcitabine, doxorubicin, bendamustin, paclitaxel, docitaxel, docetaxel, fluorouracil, imatinib mesylate, duocarmycin, irinotecan, vinblastine, sunitinib, topotecan, calicheamicin, maytansinoids, auristatins, tubulysins and analogs thereof, and various other targeted therapies approved for use in cancer patients. Chemotherapeutic agents may be administered in an antibody drug conjugate or by other targeted therapies known in the art.

In addition to serving as a cytotoxic agent, HPRN has been found to increase the sensitivity of cancer cells to chemotherapeutics. Accordingly, administration of an immunofusion molecule comprising HPRN and a chemotherapeutic agent as combination therapy may reduce the growth or size of a cancerous tumor more than administration of either the immunofusion molecule or chemotherapeutic agent alone.

### EXAMPLES

The following non-limiting Working Examples describes methods for making and using our immunofusion molecules.

### Example 1

### Synthetic Construct Generation (Antigens and Immunofusion Molecules)

Genes encoding all the proteins were codon modified for enhancement of expression in CHO cells. The synthetic coding sequences were assembled by standard molecular biology methods using Invitrogen, USA's GeneArt® gene synthesis platform. Table 1 lists the genes that were synthesized for expression.

TABLE 1
List of protein constructs generated for expression
Antigens

| | |
|---|---|
| 1 | Human 5T4 full length |
| 2 | Human 5T4-ECD-Fc |
| 3 | TAG2-ECD-Fc |
| 4 | TAG3-ECD-Fc |

Immunofusions

| | |
|---|---|
| 1 | Anti-5T4-scFv-Fc |
| 2 | anti-5T4 scFv-Fc-HPRN |
| 3 | anti-5T4 scFv-Fc-catB-HPRN-Poly E |
| 4 | anti-5T4 scFv-Fc-catB-HPRN-catB-Poly E |

pOptiVEC TOPO TA and pCDNA3.1 expression vectors were procured from Invitrogen, USA. pTT5 and pTT22 expression vectors were licensed from National Research Council (NRC), Canada. Restriction enzymes used in the study were purchased from New England Biolabs. T4 DNA Ligase and Taq DNA polymerase were procured from Bangalore Genei, India. Sequencing of constructs were carried out using Big dye terminator V 3.1 cycle sequencing kit from Applied Biosystems, USA. Gen Elute plasmid mini prep kit (Sigma, USA) and Plasmid mega kit and plasmid Giga Kits (Qiagen, USA) were used for various scales of plasmid preparation. Gen elute gel Extraction kit (Sigma, USA) was used for purification of PCR products from agarose gels. E. coli Omnimax cells used for cloning were procured from Invitrogen, USA. All methods used for cloning of the genes were based on the manufacturers' guidelines. Unless otherwise mentioned, standard molecular biology protocols (Molecular Cloning, Sambrook et al.) were followed.

The codon modified antigen sequences were cloned as their extra-cellular domain (ECD)-Fc fusion forms in two vectors—pTT5 for transient expression in HEK 293 cells and pOptiVEC TOPO for generation of stable CHO cell lines. A gene encoding 5T4 was cloned into pTT22 and pCDNA3 vectors for stable cell line generation. Codon modified immuno fusion molecules were cloned in pTT5 and pOptiVEC TOPO vectors for transient and stable expression respectively.

The cloning vector(s) were propagated in E. coli DH5α or Omnimax cells as and when required. The pTT5 vector was digested using Xba I and Not I restriction enzymes and the vector backbone was purified using a gel elution column (Sigma, USA) following manufacturer's protocol and used in subsequent cloning of both antigen ECD-Fc genes and immunofusion molecules. All constructs were transformed in E. coli DH5α or Omnimax strains and plated on LB Ampicillin agar plates followed by incubation at 37° C. for 16 h. Positive colonies were screened by colony PCR using gene specific primers. Randomly selected PCR positive colonies were inoculated in LB Ampicillin broth and plasmid DNA isolation was carried out using a mini-prep column (Sigma, USA). Isolated recombinant plasmids were subjected to restriction digestion with Xba I and Not I enzymes. Restriction positive clones were confirmed by bi-directional sequencing.

The full length (FL) 5T4 (5T4-FL) antigen gene was sub-cloned from a GeneArt® vector to a pOptiVEC vector using restriction enzymes Xba I and Not I. The positive clones were verified by restriction digestion analysis and sequencing as described above.

For subcloning into a pTT22 vector, the pOptiVEC/5T4-FL was digested using XbaI-NotI restriction enzymes and ligated to XbaI-NotI digested pTT22. For subcloning into a pCDNA 3.1 vector, the pTT22/FL 5T4 was digested with EcoRI and NotI restriction enzymes and the release insert was ligated to pcDNA 3.1 digested with identical enzymes. All constructs were sequenced and absence of any mutations was confirmed.

### Example 2

### Generation of C-terminal Glu-rich (polyE) Tail:

A Poly glutamate tail (PolyE) was added to the C-terminus of the protein. The Glu-Rich tail was designed as follows:

(SEQ ID NO: 99)
VHFDASVEDSTGLFREEEEEEASSSSSEEAEEASSSSSAEEEEGASSSEE
EASSSSAEEEEEG

The CatB amino acid sequence GLFR (SEQ ID NO:100) was inserted at the junction of HPRN and polyE tag in order for the construct to undergo intracellular proteolytic processing to release the polyE tag.

DNA encoding the polyE fragment was synthesized by assembly PCR method using overlapping primers. Purified PCR products were cloned into T/A vector followed by sequence confirmation. This fragment was attached to the C-terminal of the immunofusion molecule by overlapping PCR method. The final PCR product of the Immunofusion molecule with Poly-E tail was cloned into the pTT5 or pOptiVEC vector between the Xba I/Not I sites by restriction digestion and ligation based methods as described above.

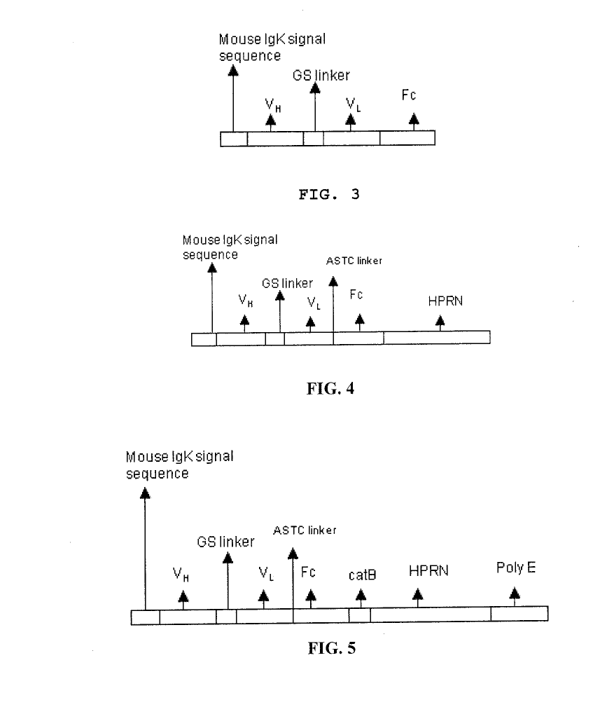

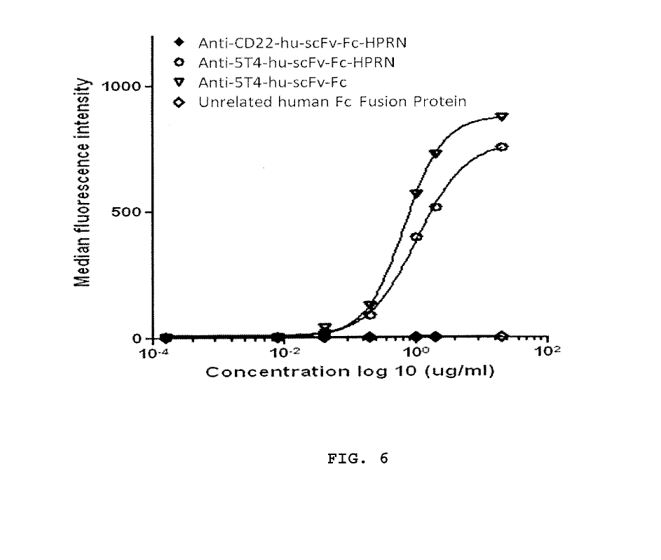

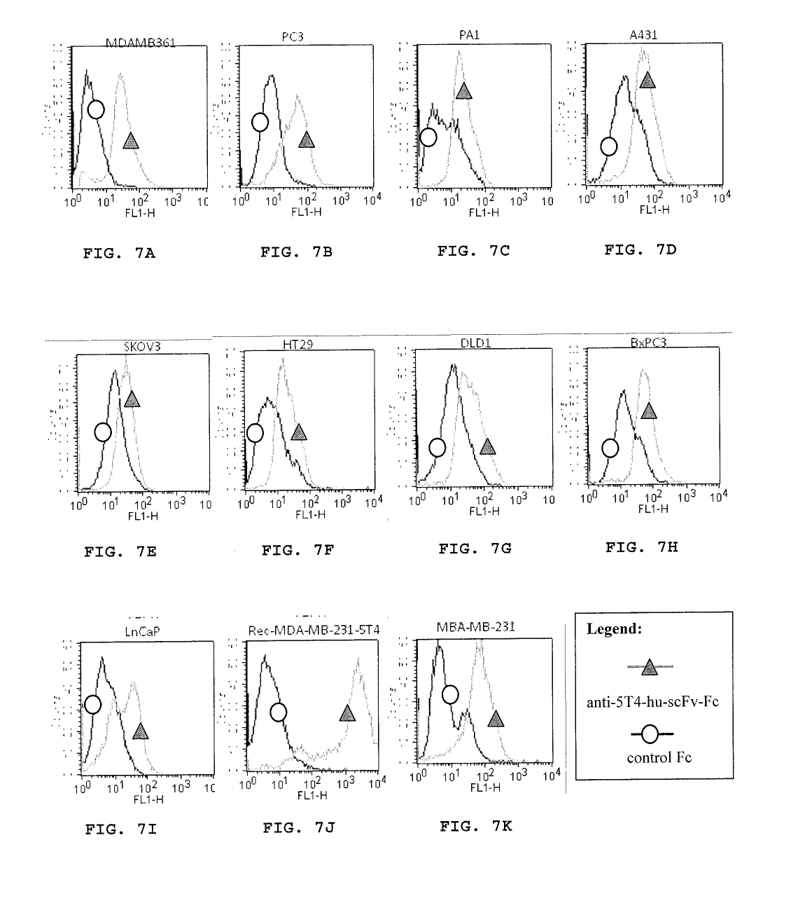

FIGS. 3 to 5 are schematic representations of the anti-5T4 immunofusion construct designs which were created and studied in the examples below. FIG. 3 is a schematic representation of an exemplary anti-5T4 scFv-Fc construct design (SEQ ID NO:101). FIG. 4 is a schematic representation of an exemplary anti-5T4 scFv-Fc-HPRN construct design (SEQ ID NO:102). FIG. 5 is a schematic representation of an exemplary anti-5T4 scFv-Fc-HPRN-PolyE construct design (SEQ ID NO:103).

### Example 3

### Transient Transfection for Protein Expression

pTT contains the Epstein-Barr virus (EBV) oriP along with an improved cytomegalovirus-based expression cassette. The HEK293-6E cell line used as an expression host harbors a truncated version of EBNA-1 protein that helps maintain the transfected plasmid as a multicopy episome.

An EBNA based transient protein expression system was licensed from NRC (National Research Council), Canada. PEI was procured from Polysciences Inc., USA. All other reagents and media were obtained from Invitrogen, USA.

The efficiency of HEK293-6E cells was determined by transfection using GFP expression vector with PEI as transfection agent. Based on the results of the trials, both antigen and antibody-Fc fusion proteins were transfected in HEK293-6E cell line at a cell density of 1.5×10⁶ cells/ml using DNA: PEI ratio of 1:3. Flasks were incubated at 37° C., 5% CO₂, at an orbital shaking speed of 100RPM. The transfections were monitored for viability and protein expression was determined using analytical protein A HPLC using PA immunodetection sensor cartridge (Applied Biosystems) attached to an Agilent 1200 HPLC.

### Example 3

### Protein Purification

MabSelect (GE Lifescience) media having a base matrix of high-flow agarose was used to purify the Fc-fusion proteins by affinity chromatography. The MabSelect matrix was packed into XK16/20 COLUMN (GE) of packed bed volume 10m1. The column was equilibrated with pre-chilled buffer A (20 mM Sodium phosphate buffer pH-7.4 & 150 mM NaCl). Prior to application to the column, the pH of the harvested culture supernatant was adjusted to 7.4 (i.e. the pH of the buffer A). The culture supernatant was passed through the pre-equilibrated column and flow-through was collected separately. The column was washed with buffer A to remove the unbound proteins and other loosely bound impurities

Bound proteins were eluted with 80 mM Acetic acid Eluted fractions were neutralized immediately after elution with 1M Tris (pH>10). Eluted protein was concentrated and buffer exchanged into buffer A+10% Glycerol using an amicon concentrator of 30 kDa cut-off. The purified proteins were analysed by SDS-PAGE, SEC and BIAcore.

### Example 4

### Stable Cell Line Generation

Two types of stable cell lines were generated. Antigen and antibody-Fc fusion proteins were made in CHO DG44 cells. Stable cell line using the full length 5T4 was generated in the cancer cell line MDA-MB-231.

cGMP banked DG44 cells (Passage 8, vial 253) procured from Invitrogen, USA were used for transfection. Prior to the transfection of DG44 cells, antigen and antibody constructs were linearized using Pvu I (NEB, USA) and purified. DG44 cells were freshly seeded at a cell density of 3×10⁵ cells/ml in 100 ml of complete DG44 medium and Erlen Meyer flasks were incubated at 37° C., 5% CO₂ at an orbital shaking speed of 130-135 RPM.

One day prior to transfection, DG44 cells were split at a seeding density of 3×10⁵ cells/ml. On the day of transfection, viable cell count was determined and 1.5×10⁷ viable cells were used for each transfection in fresh Erlen Meyer flasks containing 30 ml of CD DG44 Medium (Invitrogen, USA). To 1.2 ml of OptiPro™ SFM (serum-free, animal origin-free culture medium from Invitrogen, USA) 18 μg of linearized recombinant plasmid DNA and 15 μl of FreeStyle™ MAX reagent (transfection reagent from Invitrogen, USA) was added and mixed gently. The DNA-FreeStyle™ MAX mix was incubated for 15 minutes at room temperature to allow formation of a DNA-reagent complex. This complex was then slowly added into the 125 ml Erlen Meyer flask containing the DG44 cells. Transfected cells were incubated at 37° C., 5% CO₂ on an orbital shaker platform rotating at 130-135 rpm. After 48 h, cells were passed into CD Opti CHO media deficient in HT. Fresh media changes were given every 2 days and cultures were grown in the CD Opti CHO HT deficient media for 20 days. Cells were monitored daily for viability.

Gene amplification of the recombinant cells was carried out using methotrexate. 1 mM stock of methotrexate hydrate (Sigma, USA) was used to transfect DG44 cells grown in CD Opti CHO media deficient in HT. Cells were spun down and seeded at a density of 3×10⁵ cells/ml in Erlen Meyer flask in 30 ml of CD Opti CHO media containing 250 nM MTX. Flasks were incubated at 37° C., 5% CO2 with orbital shaking speed of 130-135 RPM. Cells were monitored daily for growth and viability and media changes were given at least 2-3 times in a week. Selection of MTX resistant cell population was done for 21 days and protein expression was analyzed using analytical protein A HPLC on the last day. Cells selected at 250 nM MTX were passaged to next round of methotrexate mediated amplification at 500 nM concentration as per the instruction in the manufacturer's manual.

A recombinant cancer cell line expressing 5T4-FL was developed for use in in vitro bioassays as well as xenograft studies as a positive control. MDA-MB-231 cells were chosen for generation of recombinant 5T4-FL clone due to the inherent low levels of 5T4 expression under normal in vitro culture conditions. MDA-MB-231 cells (ATCC, USA) were cultured in DMEM medium supplemented with 10% FBS (Thermo Scientific, USA) and 1% penicillin-streptomycin solution.

A selected concentration of the two antibiotics selection markers (G418 and Puromycin) was separately identified by kill curve assay. G418 and Puromycin were procured from Invitrogen, USA.

Two recombinant plasmids encoding 5T4-FL in pcDNA3.1 (G418) and 5T4-FL in pTT22 (Puromycin) were generated using a maxi prep plasmid extraction kit (Sigma, USA). MDA-MB 231 cells were seeded at a seeding density of 5×105cells/well in separate 6-well plates and plates were incubated for 24 h at 37° C., 5% CO₂. Transfections were carried out using Fugene 6 reagent (Roche, USA) using manufacturer's protocol. Parallel transfections were carried out for each 5T4-FL construct using DNA: Fugene 6 ratio of 1:3 and 5:2 respectively. Cells were treated either with 800 μg/ml (G418) or 0.3 μg/ml (Puromycin) after 48 h. Antibiotic selections were carried out for 15-20 days with media changes every 2-3 days. Single clones were picked by using cloning disc (Sigma, USA) and were gradually expanded. The expression of 5T4-FL was confirmed by flow cytometry.

### Example 5

### Flow Cytometry

Multi-parameter analysis of cell suspensions from different cancer cell lines was performed using a FACS Calibre flow cytometer (Beckton Dickinson) and anti-5T4 humanized-scFv-Fc (hu-scFv-Fc) fusion protein as the probe. Titration of the immunofusion protein was performed to determine the concentration of the immunofusion protein required to saturate the 5T4 antigen on the cell surface in a MDAMB 231 overexpressing 5T4 antigen.

5T4 overexpressing recombinant MDA-MB-231 cells were used for titration of anti-5T4 HPRN immunofusion proteins and anti-5T4 hu-scFv-Fc immunofusion protein. Exponentially growing cells were detached from culture flask using 0.5 mM PBS/EDTA buffer. The cells were washed twice with 1XPBS and incubated with either anti-5T4 hu scFv-Fc or unrelated Fc fusion protein (0.0005 to 100 μg/m1) in 1% BSA-1XPBS for 1 h at room temperature. The cells were washed three times with 1XPBS and incubated with anti-Fc specific-FITC antibody for 45 min at 40° C. After the incubation of anti-Fc specific FITC antibody, cells were washed three times with 1xPBS and analyzed using flow cytometry (FACS Calibre, Becton Dickinson). Median fluorescence intensities (MFI) were determined for the samples. For analysis of the wild type cancer cells, 5 μg/ml anti-5T4 hu-scFv-Fc was used and cells were processed as above. Median fluorescence intensity (MFI) was determined for each sample and compared.

### Example 6

### In vitro Cytotoxicity

Cytotoxicity assay was carried out in 96-well plates using different tumor cell lines. Five thousand exponentially growing cells were seeded in 100 μl of medium in each well of the 96-well plates. After 24 hours, cells were treated separately with different concentrations of either Anti-5T4 hu-scFv-Fc-HPRN or Anti-CD22 hu-scFv-Fc-HPRN (nonbinding negative control). 96 hours after the treatment, media with immunofusion proteins was removed from the wells, and 50 μl of CellTiter-Glo® Reagent (Luminescent Cell Viability Assay from Promega) and 50 μl of media were added to each well. Plates were incubated for 20 min and luminescene in each well was read using Hidex Chameleon plate reader.

### Example 7

### Cell Surface Expression of 5T4

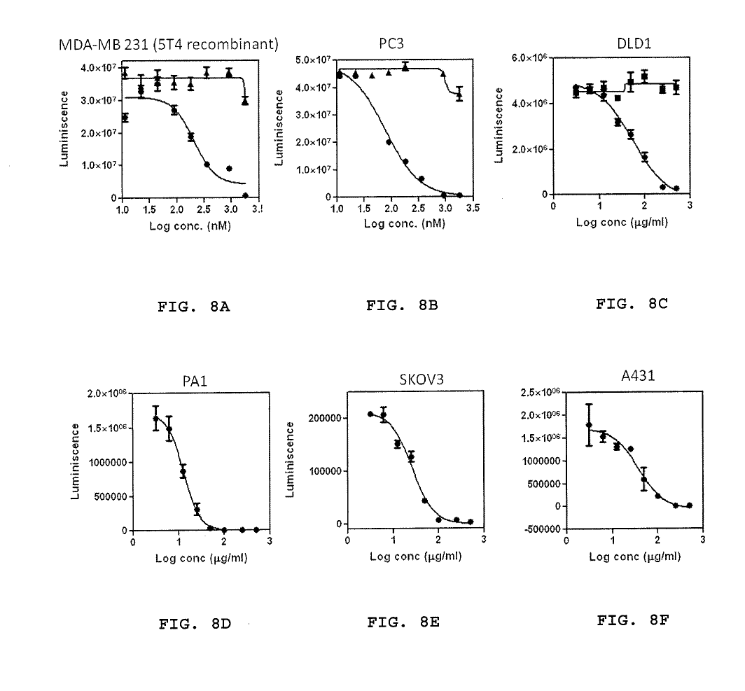

Recombinant MDA-MB-231-5T4 FL overexpressing cell surface 5T4 antigen was used to determine a saturating concentration of the Anti-5T4 scFv-Fc. A seven point dose response curve was plotted for the median fluorescence intensity at different concentrations of anti 5T4 ScFvFc. The curve is shown in FIG. 6. Specific dose-dependent cell surface binding was obtained for anti-5T4 huscFv-Fc-HPRN which was comparable to that of anti-5T4 huscFv-Fc. Binding was saturated at 5 μg/ml concentration. Unrelated Fc Fusion protein binding was 200 fold less at 5 μg/ml (FIG. 6).

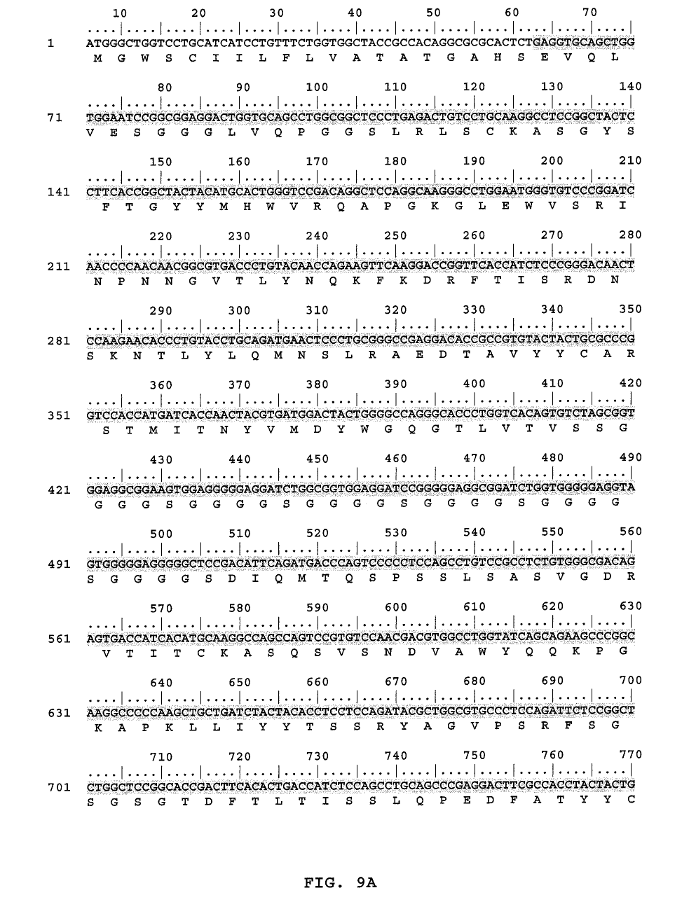

Eleven cancer cell lines derived from different types of tumors were assessed for cell surface expression of 5T4 (FIGS. 7A-7K). The cell lines were MDAMB 361 (FIG. 7A), PC3 (FIG. 7B), PA1 (FIG. 7C), A431 (FIG. 7D), SKOV3 (FIG. 7E), HT29 (FIG. 7F), DLD1 (FIG. 7G), BxPC3 (FIG. 7H), LnCaP (FIG. 7I), Rec-MDA-MB-231-5T4 (FIG. 7J) and MBA-MB-231 (FIG. 7K). The flow cytometric analysis of 5T4 expression in the cancer cell lines is shown by histograms representing fluorescence profiles obtained using anti-5T4-hu-scFv-Fc (gray line with triangle) and an unrelated (control) Fc fusion protein (black line with circle) as probes. The median fluorescence intensities (MFI) for each line are summarized in Table 2.

TABLE 2
Summary of 5T4 expression analysis in cancer cell lines by
Flowcytometry

| | Median fluorescence intensities (MFI) | |
|---|---|---|
| | anti-5T4-hu-scFv-Fc | unrelated fusion protein |
| Cell Line | (5 μg/ml) | (5 μg/ml) |
| MDAMB361 | 37 | 5 |
| PC3 | 41 | 3 |
| PA1 | 20 | 6 |
| A431 | 26 | 13 |
| SKOV3 | 17 | 6 |
| HT29 | 33 | 12 |
| DLD1 | 50 | 14 |
| BxPC3 | 46 | 14 |
| LNCaP | 18 | 23 |
| Rec-MDA-MB-231-5T4 | 27 | 8 |
| MBA-MB-231 | 1655 | 4 |

All cell lines expressed 5T4 antigen to different extents. MDAMB 361, BXPC3 and A431 showed the highest expression of 5T4 antigen, while LNCaP and HT 29 were weakly positive.

### Example 7

### In vitro Cytotoxicity

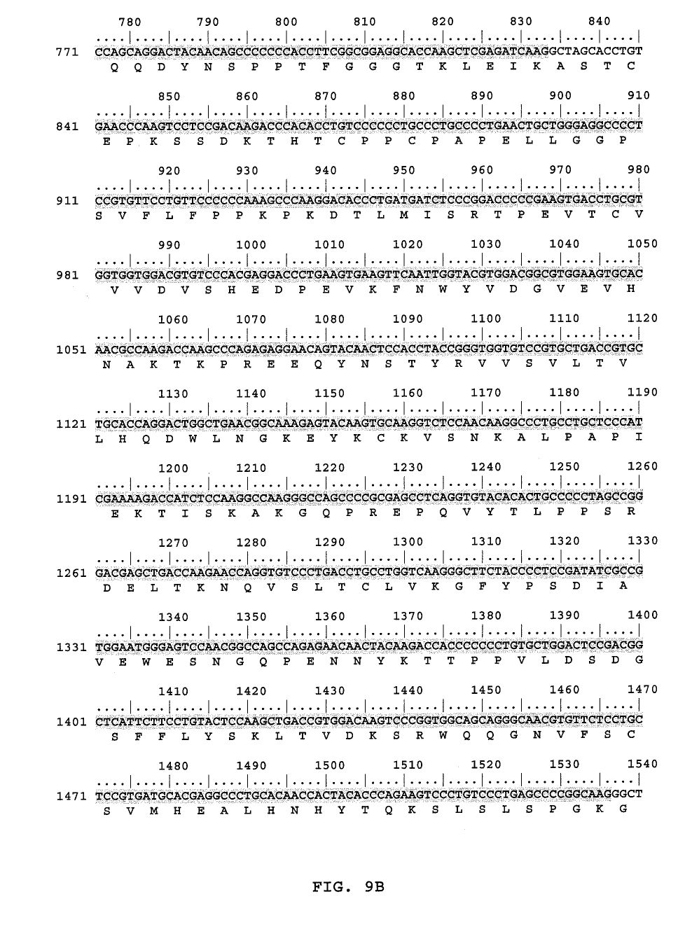

Detection of cell proliferation to determine genotoxicity, and evaluating anticancer drugs or antibodies is a fundamental method for assessing cell health. The CellTiter-Glo® Luminescent Cell Viability Assay, which quantitates the ATP present (presence of metabolically active cells), was used to determine the number of viable cells in culture. When examined for the growth inhibitory effect of HPRN immunofusion proteins against a panel of human carcinoma cell lines, anti-5T4 scFv-Fc-HPRN but not anti-CD22 scFv-Fc-HPRN caused a dose-dependent inhibition of growth of human tumor cells (FIGS. 8A-8F).

The tumor cells studied where Recombinant MDA-MB-231-5T4 (FIG. 8A), PC3 (FIG. 8B), DLD1 (FIG. 8C), PA1(FIG. 8D), SKOV3 (FIG. 8E), A431(FIG. 8F). Each of these tumor cells was shown to express 5T4 on their surface as determined by flow cytometry (FIGS. 7B, C, D, E, G and J, above). The dose dependent cytotoxicity of anti-5T4-humanized scFv-Fc-HPRN (circles) was determined by the ATP Glo method. Anti CD-22 humanized scFv-Fc (triangle or square) was used as negative control. Curves were generated by four parameter non-linear regression analysis using GraphPad Prism. Table 3 summarizes the results for the cytotoxicity of Anti-5T4 hu-scFv-Fc-HPRN.

TABLE 3
Summary of cytotoxicity of Anti-5T4 huscFv-Fc-HPRN

| | | IC₅₀ |
|---|---|---|
| Cell lines | IC₅₀ (μg/ml) | (nM) using tetramer MW |
| A431 | 38 | 135 |
| DLD1 | 55-63 | 195-222 |
| SKOV3 | 27 | 92 |
| PC3 | 5-11 | 19-39 |
| PA-1 | 10-16 | 34-55 |
| MDA MB-231 5T4 transfectant | 60-85 | 213-300 |

### Example 8

### Pharmacokinetic Estimation of Anti-5T4-HPRN Immunofusion Proteins in Mice by Enzyme Linked Immunosorbent Assay (ELISA)

The pharmacokinetic properties of the anti-5T4 immunofusion proteins were estimated by measuring the blood immunofusion protein concentration at various time points after intravenous injection in nude mice. The anti-5T4 scFvFc protein concentration in the blood was estimated by BIAcore while the concentration of immunofusion proteins (anti-5T4 scFvFc HPRN and its variants) was estimated using an indirect ELISA. This is because the immunofusion protein is a unit containing two functional domains, an antigen binding domain and the RNAse domain and the BIAcore method would only detect the antigen binding domain. Any BIAcore detection would limit the detection to the antigen binding domain only, while it is desired to detect the intact ImmunoRNAse in the blood for accurate pharmacokinetic estimation.

In the BIAcore procedure, the affinity of anti5T4 immunofusion proteins to 5T4 was determined by SPR analysis using a BIAcore T200 (GE Healthcare). Briefly, purified 5T4-extracellular domain-human Fc fusion protein was covalently immobilized on a BIAcore CM5 sensor chip by amine coupling method using reagents and instructions provided by the manufacturer. In the binding study, anti-5T4 immunofusion proteins were serially diluted to a concentration series and flowed over the immobilized antigen for a fixed period of time, followed by flow of buffer to dissociate the antigen. At the end of the dissociation cycle, regeneration of chip the surface was carried out at low pH. The resulting sensorgrams were fit to a 1:1 Langmuir binding model using the BIAevaluation software (GE Healthcare) and the kinetics parameters like association rate, dissociation rate and affinity were estimated.

Diluted mouse blood (typically 40-fold) from various time points were flowed over the surface and the response recorded. A standard curve was constructed using a concentration series of the protein in non-immunized mouse blood diluted 40-fold flowed over the sensor chip. The unknown concentrations were calculated by interpolation from the standard curve.

In the ELISA method, the 5T4-ECD-Fc antigen is used to capture the immunofusion protein from diluted mouse blood (typically 400-fold), followed by binding of Anti-RNAse antibody to captured immunofusion protein. The complex is then detected using a commercial secondary antibody reactive to the Anti-RNAse antibody. A standard curve is constructed using a concentration series of the immunofusion protein in non-immunized mouse blood in the same ELISA as the sample. The unknown concentrations were calculated by interpolation from the standard curve.

The blood concentration versus time profile was analyzed using a non-compartmental model and the pharmacokinetic parameters like half-life, bioavailability and elimination rate were calculated.

The anti-5T4 scFvFc, anti-5T4 scFvFc-HPRN, and anti-5T4 scFvFc-HPRN PolyE immunofusion proteins were injected intravenously into nude mice (n=3) at the doses ranging from 10 to 30 mg/kg. The blood concentrations were measured as mentioned above.

The results of the pharmacokinetic assessment of these immunofusions are summarized in Table 4.

TABLE 4
Pharmacokinetics

| Immunofusion | t_{1/2} | C₀ | AUC ₍₀₋ₜ₎ | AUC _{(0-∞)} | V_{D} | C_{L} | MRT |
|---|---|---|---|---|---|---|---|
| Unit | (hr) | μg/ml | μg/ml * hr | μg/ml * hr | L/kg | mL/min/kg | (hr) |
| Anti-5T4 scFvFc | 56.3 | 131 | 5543 | 7743 | 0.1 | 0.02 | 90 |
| 10 mg/kg | | | | | | | |
| Anti-5T4 | 19.7 | 52 | 192 | 325 | 0.9 | 0.5 | 26 |
| scFvFc-HPRN | | | | | | | |
| 10 mg/kg | | | | | | | |
| Anti-5T4 | 19.1 | 221 | 515 | 813 | 1.0 | 0.6 | 24 |
| scFvFc-HPRN | | | | | | | |
| 30 mg/kg | | | | | | | |
| Anti-5T4 | 29 | 171 | 2096 | 4637 | 0.3 | 0.1 | 41 |
| scFvFc-HPRN PolyE | | | | | | | |
| 30 mg/kg | | | | | | | |
| Anti-5T4 | 21 | 87 | 950 | 1770 | 0.2 | 0.1 | 30 |
| scFvFc-HPRN PolyE | | | | | | | |
| 10 mg/kg | | | | | | | |

### Example 9

### In vivo Efficacy Evaluation in Xenograft Models:

### Materials

BD 1 ml syringes (271/2 Gauge), Sterile Culture medium, Sterile Phosphate Buffered Saline, Matrigel-BD Biosciences (catalog No. 354248), Sterile cotton plugs, Sterile Eppendrof tubes (1.5 mL, 2 mL), Pipettes, Filter paper, 70% Alcohol/Isopropyl alcohol, Vernier Caliper (Mitutoyo). All other essential items used were of analytical grade.

### Animals

Athymic male & female nude mice (Hsd: Athymic Nude-Foxnlnu) 5-6 weeks old, weighing 20-22 g were obtained from Harlan, Netherlands Animals were taken care as per the Regulations of Committee for the Purpose of Control and Supervision of Experiments on Animals (CPCSEA), Government of India and Association for Assessment and Accreditation of Laboratory Animal Care (AAALAC) compliance. The ‘Form B’ for carrying out animal experimentation was reviewed and approved by the Institutional Animal Ethics Committee (IAEC Protocol Approval No: SYNGENE/IAEC/160/09-2010).

### Housing and Feeding

Animals were maintained in a controlled environment with 22±3° C. temperature, 50±20% humidity, a light/dark cycle of 12 hours each and 15-20 fresh air changes per hour. Animals were housed group wise and autoclaved corncob was used as a bedding material. The animals were fed, ad libitum, with certified Irradiated Laboratory Rodent Diet during the study period.

### Preparation of Animals & Animal Identification

The animals were kept under acclimatization in the experimental room for a period of at least 5 days. Animals were individually numbered and the cage cards indicating the experiment, study number, date of tumor implantation, date of randomization, tumor type, mouse strain, gender, and individual mouse number were displayed to corresponding cages. After randomization, group identity, test compound, dosage, schedule and route of administration were added.

### Preparation of Tumor Cells

All procedures were performed in laminar flow hood following sterile techniques. Cancer cells (A431 (Epidermoid), PA-1 (Ovarian), PC-3 (Prostate) & LLC (Lewis lung carcinoma)) with 70-80% confluent and viability of >90% was chosen for the study. Ideally 5×10⁶ cells (A431, PA-1, PC-3 & LLC) was resuspended in 200 μl of PBS or serum free media containing 50% of matrigel kept in ice.

### Subcutaneous Injection of Cells

Nude mice (Hsd: Athymic Nude-Foxnlnu) housed in Individual Ventilated Cages (IVCs) was used for the investigation. Cancer cell lines (A431, PA-1, PC-3 & LLC) were propagated in the animals by injecting the cancer cells subcutaneously in the flanks or back of the animals. The implanted area was monitored for growth of tumor. Once the tumor attained palpable and required volume (TV≈100-150 mm³), animals were randomized based on tumor volume and dosing was initiated. The tumor volume was determined by two-dimensional measurement with a caliper on the day of randomization (Day 0) and then once every three days (i.e. on the same days on which mice were weighed). Using a vernier caliper the length (l) and width (w or b) of the tumor was measured. Tumor volume (TV) was calculated using the following formula:

Tumor Volume (mm³)=l•W²/2,

where, l=Length (mm); W=Width (mm)

In general, all the immunofusion antibodies were dissolved in sterile 1x PBS which resulted in clear solutions at all prepared concentrations. The test item was freshly prepared on the days of administration and the dose volume was kept at 10 ml/kg body weight. For each group separate new syringe and needles were used.

### Body Weight

Cage side observations, body weight were measured once every three days during the study period. The % change in body weights of individual mice was calculated.

### Collection of Blood

Approximately 5 μL blood sample was collected at 0 min (prior application), 5 min, 15 min, 30 min, 60 min, 180 min, 360 min, 24 h, 48 h, 72 h, 96 h, 120 h, 144 h and 168 h post application. For each time point, the sample was collected by tail vein puncture and immediately transferred to a centrifuge tube containing 195 μL of diluent buffer (PBS containing EDTA, 2 mg/ml of blood). The samples were immediately transferred to a box containing crushed ice. Blood samples were centrifuged at 2500×g, 4° C. for 5 minutes. The resultant supernatants were transferred into new tubes and were subjected to PK analysis.

### Antitumor Activity

Antitumor activity was evaluated as maximum tumor volume inhibition versus the vehicle control group. Data evaluation was performed using statistical software Graph pad version.5.

### Test/Control Value in % (% T/C)

Tumor inhibition on a particular day (T/C in %) was calculated from the ratio of the mean TV values of the test versus control groups multiplied by 100%.

$$T / C ( {Day}_{x} ) = \frac{Mean tumor volume of the test group {Day}_{x}}{Mean tumor volume of the control group {Day}_{x}} \times 100 \%$$

The minimum (or optimum) T/C % value recorded for a particular test group during an experiment represents the maximum antitumor activity for the respective treatment. Tumor growth inhibition (TGI)

TGI was calculated using the following formula:

TGI=(1-T/C)×100

Where, T=mean tumor volume in the treated group; C=mean tumor volume in the vehicle control group on a given day.

### Clinical Signs: Morbidity & Mortality

Animals were observed individually for visible general clinical signs once every three days during the study period. All the animals were checked for morbidity and mortality.

### Statistical Analysis

For the evaluation of the statistical significance of tumor inhibition, Two-way ANOVA followed by Bonferroni post-test was performed using GraphPad Prism v5. p values <0.05 indicate statistically significant differences between groups.

### Results

A431 Subcutaneous Xenograft

Antitumor Activity

A431 xenograft bearing mice were treated with Anti-5T4 scFv-Fc HPRN (Loading dose: 30 mg/kg, i.v on Days 0, 2, 4, 6 & 9; followed by maintenance dose of 15 mg/kg, i.v on Days 11, 13, 16, 18 & 20) ImmunoRNase therapy demonstrated moderate antitumor activity against A431 xenograft tumor model. Treatment with either anti-5T4 scFv-Fc HPRN or anti-5T4 scFv-Fc resulted in an optimal T/C value of 52.7% and 85.4% respectively on Day 20. The % tumor growth inhibition (TGI) for Anti-5T4 scFv-Fc HPRN group at the tested dose level was found to be 47.3% (Day 20, p<0.001). The % TGI for Anti-5T4 scFv-Fc group was 14.64% (Day 20) which was statistically non-significant.

Mortality and Body Weight Changes

There was no body weight loss in Vehicle control & Anti-5T4 scFv-Fc treated group during the experiment period. All animals were active and healthy. Anti-5T4 scFv-Fc HPRN therapy was relatively well tolerated at the tested dose level with no mortality. Moreover, there were no visible signs of abnormal behavior or any adverse clinical symptoms during treatment.

Additional A431 Subcutaneous Xenograft

Antitumor Activity

In a subsequent xenograft with the A431 cell line, Anti-5T4 scFv-Fc HPRN was administered at a dose of 30 mg/kg, i.v; QDx11, to nude mice bearing subcutaneous epidermoid carcinoma (A431) tumor xenografts. The dosing regimen used in the second study resulted in a better tumor growth inhibition. Treatment with anti-5T4 scFv-Fc HPRN resulted in an optimal T/C of 36.9% on Day 18.

Mortality and Body Weight Changes

Anti-5T4 scFv-Fc HPRN was relatively well tolerated at the tested dose level with no mortality. There was no significant body weight loss in Vehicle control & Anti-5T4 scFv-Fc HPRN treated group during the experiment period. All animals were active and healthy. Based on cage side observations there was no visible signs of abnormal behavior or clinical symptoms in Anti-5T4 scFv-Fc HPRN treated group.

Lewis Lung Carcinoma (LLC) Subcutaneous Xenograft

Antitumor Activity

A murine Lewis Lung Carcinoma (LLC) xenograft was used to examine the specificity of the antibody. Anti-5T4 scFv-Fc HPRN was administered at the dose of 30 mg/kg, i.v; QDx10, to nude mice bearing subcutaneous LLC tumor xenografts. Administration of Anti-5T4 scFv-Fc HPRN once daily for 10 days to nude mice bearing subcutaneous LLC tumors at the tested dose did not cause any significant % reduction in tumor volume of LLC xenograft. The % T/C value of on Day 18 was found to be 92.4%. The difference in tumor sizes between the control group and the treatment group was not statistically significant and the % tumor growth inhibition (TGI) at this dose was found to be 7.6% (Day 18).

Mortality and Body Weight Changes

Anti-5T4 scFv-Fc HPRN was relatively well tolerated at the tested dose level with no mortality. There was no body weight loss in Vehicle control & transient body weight loss in Anti-5T4 scFv-Fc HPRN treated group during the experiment period. All animals were active and healthy. Based on cage side observations there was no visible signs of abnormal behavior or clinical symptoms in Anti-5T4 scFv-Fc HPRN treated group.

Ovarian Teratocarcinoma (PA-1) Subcutaneous Xenograft

Antitumor Activity

In a xenograft experiment with the PA-1 cell line, Anti-5T4 scFv-Fc HPRN was administered at a dose of 30 mg/kg, i.v; QDx10, to nude mice bearing subcutaneous ovarian teratocarcinoma (PA-1) tumor xenografts ImmunoRNase therapy demonstrated moderate antitumor activity against PA-1 xenograft tumor model. Treatment with either anti-5T4 scFv-Fc HPRN or anti-5T4 scFv-Fc resulted in an optimal T/C value of 53.3% and 87.5% respectively on Day 21. The % tumor growth inhibition (TGI) for Anti-5T4 scFv-Fc HPRN group at the tested dose level was found to be 46.68% (Day 21, p<0.001). The % TGI for Anti-5T4 scFv-Fc group was 12.49% (Day 21) which was statistically non-significant.

Mortality and Body Weight Changes

Anti-5T4 scFv-Fc HPRN was relatively well tolerated at the tested dose level with no mortality. There was no body weight loss in Vehicle control & transient body weight loss in Anti-5T4 scFv-Fc HPRN treated group during the experiment period. All animals were active and healthy. Based on cage side observations there was no visible signs of abnormal behavior or clinical symptoms in Anti-5T4 scFv-Fc HPRN treated group.

Prostate Cancer (PC-3) Subcutaneous Xenograft

Antitumor Activity

In a xenograft experiment with the PC-3 cell line, Anti-5T4 scFv-Fc HPRN was administered at a dose of 15 mg/kg, i.v; QDx9, to nude mice bearing subcutaneous prostate cancer (PC-3) tumor xenografts ImmunoRNase therapy demonstrated moderate antitumor activity against PC-3 xenograft tumor model. Treatment with either anti-5T4 scFv-Fc HPRN or anti-5T4 scFv-Fc resulted in an optimal T/C value of 64.3% and 88.2% respectively on Day 21. The % tumor growth inhibition (TGI) for Anti-5T4 scFv-Fc HPRN group at the tested dose level was found to be 35.74% (Day 21, p<0.001). The % TGI for Anti-5T4 scFv-Fc group was 11.75% (Day 21) which was statistically non-significant.

Mortality and Body Weight Changes

Anti-5T4 scFv-Fc HPRN was relatively well tolerated at the tested dose level with no mortality. There was no body weight loss in Vehicle control & mild body weight loss in Anti-5T4 scFv-Fc HPRN treated group during the experiment period. All animals were active and healthy. Based on cage side observations there was no visible signs of abnormal behavior or clinical symptoms in Anti-5T4 scFv-Fc HPRN treated group.

IHC Materials and Methods

Tumor tissues were harvested at different stages and were fixed in 10% buffered neutral formalin for 24 to 48 hours (Biochain, USA, Cat. No T2234200). Human placental uterus biopsy tissues were obtained from local hospital and were simultaneously processed for paraffin embedding. The sections obtained from these tissues were used as positive and negative controls, respectively in the immunohistochemical localization of 5T4 antigen. Tumor tissues were processed for paraffin embedding, paraffin-embedded tissues were sectioned at 5 μm thickness and mounted on glass slides.

Mounted sections were deparaffinized in xylene and gradually hydrated using descending alcohol grades followed by washes in distilled water and PBS. Immunohistochemical localization of 5T4 was carried out using R&D system kit. A set of tissue sections were also stained with hematoxylin and eosin for histological examination.

The immunohistochemical localization of 5T4 antigen in various tumor and human placenta tissues was performed as per the manufacturer's instructions (R&D system).

Briefly, the deparaffinized and hydrated tissue sections were processed for antigen retrieval by incubating slides at 95° C. for 10 min in antigen retrieval reagent (cat. No S013, R&D system). Endogenous peroxidase activity was blocked with peroxidase blocking reagent from Cell & Tissue staining kit (Cat. No. CTS019, R&D system). Subsequently, the sections were incubated in steps with serum blocking, avidin blocking and biotin blocking reagents. These blocked sections were incubated overnight at 4° C. with primary antibody [sheep polyclonal anti-human 5T4 antibody (Cat. No.: AF4975, R&D system)] followed by washing with PBS and incubation with donkey anti-sheep antibody (R&D system). The tissue sections were incubated with high sensitivity streptavidin-HRP conjugate (HSS-HRP, R& D System). The DAB Chromogen substrate was used to develop the HRP labeling followed by counterstaining using hematoxylin. The slides were mounted using DPX mountant.

Placental tissues were used to optimize the IHC procedure. The primary antibody was used at 2.5 μg/ml, 5 μg/ml and 10 μg/ml concentration to get optimum intensity of 5T4 localization. Null control (no primary antibody) was also used as a negative control at the time of standardization of IHC protocol.

Primary antibody at 5 μg/ml concentration showed optimum staining of 5T4 antigen. The concentrations 2.5 μg/ml and 10 μg/ml, showed mild and severe intensity of staining, respectively. The negative control, human uterine tissue section did not show any staining corresponding to 5T4 antigen.

The immunolocalization of 5T4 in these tumor tissues was qualitatively assessed as mild (+), moderate (++), marked (+++) and severe (++++) and its distribution was categorized into focal, multifocal and diffuse. Table 5 shows the results of immunolocalization of the 5T4 expression in xenograft tumors of the cell lines A431, DLD1, BXPC3 and PA1.

TABLE 5
Summary of 5T4 expression analysis in xenograft tumors

| Cell | Early Stage Tumor | Mid Stage Tumor | Late Stage Tumor |
|---|---|---|---|
| Lines | (150 mm³) | (500 mm³) | (1000 mm³) |
| A431 | ++ | +++ | ++++ |
| | diffuse | multifocal | diffuse |
| DLD1 | ++ | Negative | Very mild diffuse |
| | diffuse | | |
| BXPC3 | ++ | +++ | ++++ |
| | diffuse | diffuse | diffuse |
| PA-1 | ++ | +++ | ++++ |
| | diffuse | multifocal | multifocal |

### Example 10

### Bladder Installation Procedure

A pharmaceutical composition comprising immunofusion molecules may be prepared with saline such that the final solution has a concentration of anti-5T4 immunofusion molecules of about 5 μM, 1 μM, 0.1 μM or 0.05 μM.

A 18 or 20 F three-way Foley catheter may be inserted through the urethra and into the bladder of a patient suffering from bladder cancer and the catheter balloon may be inflated. The residual urine may be emptied. An infusion of saline at body temperature may be used to irrigate the bladder and the saline may be drained.

Up to 100 mL the pharmaceutical composition may be introduced into the emptied bladder, retained in the bladder for 30 minutes, then the bladder may be emptied and rinsed with normal saline. Alternatively, the pharmaceutical composition may be introduced into the bladder and allowed to reside there until the patient urinates.

All publications, patent application publications and issued patents cited above are incorporated herein by reference.

Although specific examples have been shown and described herein for purposes of illustration and exemplification, it is understood by those of ordinary skill in the art that the specific examples shown and described may be substituted for a wide variety of alternative and/or equivalent implementations without departing from the scope our methods, molecules and compositions. This disclosure is intended to cover any adaptations or variations of the examples discussed herein.

What is claimed is:

1. An immunofusion molecule comprising an antigen-binding portion and an RNase portion in a single chain peptide, wherein:
a) the polypeptide sequence of the antigen-binding portion comprises SEQ ID NO:22, SEQ ID NO:23, SEQ ID NO:97 or SEQ ID NO:98, and
b) the polypeptide sequence of the RNase portion comprises any one of SEQ ID NO:13 to SEQ ID NO:20.

2. The immunofusion molecule of claim 1, wherein the antigen-binding portion and RNase portion are fused by a polypeptide linker.

3. The immunofusion molecule of claim 2, wherein the polypeptide linker has a polypeptide sequence according to any one of SEQ ID NO:9 to SEQ ID NO:12.

4. The immunofusion molecule of claim 1, further comprising a CathepsinB substrate sequence fused to N-terminus or C-terminus of the RNase portion.

5-6. (canceled)

7. The immunofusion molecule of claim 4, further comprising at least ten glutamate residues fused to the C-terminus of the CathepsinB substrate sequence.

8. (canceled)

9. An immunofusion molecule comprising an antigen-binding portion and a RNase portion in a single-chain peptide, wherein the polypeptide sequence of the single-chain peptide comprises the polypeptide sequence according to any one of SEQ ID NO:32 to SEQ ID NO:95.

10. The immunofusion molecule of claim 1, wherein the single chain peptide further comprises an N-terminal signal sequence designated as SEQ ID NO:2.

11. The immunofusion molecule of claim 1, wherein the single-chain peptide forms a monomer of a dimer or a tetramer upon expression in a cell.

12-13. (canceled)

14. The immunofusion molecule of claim 9, wherein the polypeptide sequence of the single-chain peptide is modified by insertion, substitution or deletion of one or more amino acids and the immunofusion molecule binds the 5T4-antigen.

15. An isolated polynucleotide encoding a single-chain peptide comprising the polypeptide sequence according to any one of SEQ ID NO:32 to SEQ ID NO:95.

16-25. (canceled)

26. A method of treating a disease or disorder involving the expression of the 5T4 antigen comprising administering to an animal in need of such treatment a therapeutically effective amount of an immunofusion molecule comprising an antigen-binding portion and a RNase portion in a single chain peptide, wherein the polypeptide sequence of the antigen-binding portion comprises SEQ ID NO:22, SEQ ID NO:23, SEQ ID NO:97 or SEQ ID NO:98, and the polypeptide sequence of the RNase portion comprises any one of SEQ ID NO:13 to SEQ ID NO:20.

27. The method of claim 26, wherein the disease or disorder is cancer selected from the group consisting of colorectal cancer, bladder cancer, gastric cancer, breast cancer, lung cancer and prostate cancer.

28-32. (canceled)

33. An isolated polypeptide designated as SEQ ID NOs:21, 22, 97 or 98.

34. An isolated polypeptide designated as SEQ ID NOs:101, 102 or 103.

35. An immonufusion molecule comprising SEQ ID NO:103.


SEQUENCE LISTING (113 sequences)

SEQ ID NO: 1 - protein, 654 aa, Artificial Sequence
  immunofusion peptide
MGWSCIILFLVATATGAHSEVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGXSGGPGGGSKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTKEDL

SEQ ID NO: 2 - protein, 19 aa, Artificial Sequence
  signal peptide
MGWSCIILFLVATATGAHS

SEQ ID NO: 3 - protein, 4 aa, Artificial Sequence
  ER localization signal
KEDL

SEQ ID NO: 4 - protein, 242 aa, Artificial Sequence
  single chain ScFv 5T4-specific murine
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSGGGGSGGGGSGGGGSSIVMTQTPTSLLVSAGDRVTITCKASQSVSNDVAWYQQKPGQSPKLLISYTSSRYAGVPDRFTGSGSGTDFTLTISSVQAEDAAVYFCQQDYNSPPTFGGGTKLEIK

SEQ ID NO: 5 - protein, 257 aa, Artificial Sequence
  ScFv 5T4-specific humanized (human VH3/VK3 frameworks)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIK

SEQ ID NO: 6 - protein, 4 aa, Artificial Sequence
  Site-specific conjugation-1 linker (ASTC)
ASTC

SEQ ID NO: 7 - protein, 4 aa, Artificial Sequence
  No site-specific conjugation-1 linker (ASTX)
ASTX

SEQ ID NO: 8 - protein, 232 aa, Artificial Sequence
  Human Hinge-CH2-CH3 (Fc 1)
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 10 aa, Artificial Sequence
  Fc-RNase linker (GC)
GCSGGPGGGS

SEQ ID NO: 10 - protein, 10 aa, Artificial Sequence
  Fc-RNase linker (GS)
GSSGGPGGGS

SEQ ID NO: 11 - protein, 14 aa, Artificial Sequence
  Fc-RNase linker (GC/CatB)
GCSGGPGGGSGLFR

SEQ ID NO: 12 - protein, 14 aa, Artificial Sequence
  Fc-RNase linker (GS/CatB)
GSSGGPGGGSGLFR

SEQ ID NO: 13 - protein, 128 aa, Homo sapiens
  MISC_FEATURE (1)..(128): Pancreatic Ribonuclease (HPRN)
KESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 14 - protein, 128 aa, Homo sapiens
  MISC_FEATURE (1)..(128): Pancreatic Ribonuclease
KESRAKKFQRQHMDSDSSPSSSSTYCNLMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 15 - protein, 128 aa, Homo sapiens
  MISC_FEATURE (1)..(128): Pancreatic Ribonuclease
KESRAKKFQRQHMDSDSSPSSSSTYCNQMMCCRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 16 - protein, 128 aa, Homo sapiens
  MISC_FEATURE (1)..(128): Pancreatic Ribonuclease
KESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 17 - protein, 128 aa, Homo sapiens
  MISC_FEATURE (1)..(128): Pancreatic Ribonuclease
KESRAKKFQRQHMDSDSSPSSSSTYCNLMMCCRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 18 - protein, 128 aa, Homo sapiens
  MISC_FEATURE (1)..(128): Pancreatic Ribonuclease
KESRAKKFQRQHMDSDSSPSSSSTYCNQMMCCRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 19 - protein, 128 aa, Homo sapiens
  MISC_FEATURE (1)..(128): Pancreatic Ribonuclease
KESRAKKFQRQHMDSDSSPSSSSTYCNLMMRRRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 20 - protein, 128 aa, Homo sapiens
  MISC_FEATURE (1)..(128): Pancreatic Ribonuclease
KESRAKKFQRQHMDSDSSPSSSSTYCNLMMCCRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 21 - protein, 478 aa, Artificial Sequence
  Chimeric anti-5T4 scFv-Fc
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSGGGGSGGGGSGGGGSSIVMTQTPTSLLVSAGDRVTITCKASQSVSNDVAWYQQKPGQSPKLLISYTSSRYAGVPDRFTGSGSGTDFTLTISSVQAEDAAVYFCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 493 aa, Artificial Sequence
  Humanized anti-5T4 scFv-Fc (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 493 aa, Artificial Sequence
  Humanized anti-5T4 scFv-Fc (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 503 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGS

SEQ ID NO: 25 - protein, 503 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGS

SEQ ID NO: 26 - protein, 507 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSGLFR

SEQ ID NO: 27 - protein, 507 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSGLFR

SEQ ID NO: 28 - protein, 503 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGS

SEQ ID NO: 29 - protein, 503 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGS

SEQ ID NO: 30 - protein, 507 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSGLFR

SEQ ID NO: 31 - protein, 507 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSGLFR

SEQ ID NO: 32 - protein, 631 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 33 - protein, 631 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 34 - protein, 635 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSGLFRKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 35 - protein, 635 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSGLFRKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 36 - protein, 631 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 37 - protein, 631 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 38 - protein, 635 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSGLFRKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 39 - protein, 635 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSGLFRKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 40 - protein, 631 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSKESRAKKFQRQHMDSDSSPSSSSTYCNLMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 41 - protein, 631 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSKESRAKKFQRQHMDSDSSPSSSSTYCNLMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 42 - protein, 635 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSGLFRKESRAKKFQRQHMDSDSSPSSSSTYCNLMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 43 - protein, 635 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSGLFRKESRAKKFQRQHMDSDSSPSSSSTYCNLMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 44 - protein, 631 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSKESRAKKFQRQHMDSDSSPSSSSTYCNLMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 45 - protein, 631 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSKESRAKKFQRQHMDSDSSPSSSSTYCNLMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 46 - protein, 635 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSGLFRKESRAKKFQRQHMDSDSSPSSSSTYCNLMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 47 - protein, 635 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSGLFRKESRAKKFQRQHMDSDSSPSSSSTYCNLMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 48 - protein, 631 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSKESRAKKFQRQHMDSDSSPSSSSTYCNQMMCCRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 49 - protein, 631 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSKESRAKKFQRQHMDSDSSPSSSSTYCNQMMCCRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 50 - protein, 635 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSGLFRKESRAKKFQRQHMDSDSSPSSSSTYCNQMMCCRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 51 - protein, 635 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSGLFRKESRAKKFQRQHMDSDSSPSSSSTYCNQMMCCRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 52 - protein, 631 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSKESRAKKFQRQHMDSDSSPSSSSTYCNQMMCCRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 53 - protein, 631 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSKESRAKKFQRQHMDSDSSPSSSSTYCNQMMCCRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 54 - protein, 635 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSGLFRKESRAKKFQRQHMDSDSSPSSSSTYCNQMMCCRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 55 - protein, 635 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSGLFRKESRAKKFQRQHMDSDSSPSSSSTYCNQMMCCRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 56 - protein, 631 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 57 - protein, 631 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 58 - protein, 635 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSGLFRKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 59 - protein, 635 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSGLFRKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 60 - protein, 631 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 61 - protein, 631 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 62 - protein, 635 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSGLFRKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 63 - protein, 635 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSGLFRKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 64 - protein, 631 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSKESRAKKFQRQHMDSDSSPSSSSTYCNLMMCCRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 65 - protein, 631 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSKESRAKKFQRQHMDSDSSPSSSSTYCNLMMCCRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 66 - protein, 635 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSGLFRKESRAKKFQRQHMDSDSSPSSSSTYCNLMMCCRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 67 - protein, 635 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSGLFRKESRAKKFQRQHMDSDSSPSSSSTYCNLMMCCRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 68 - protein, 631 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSKESRAKKFQRQHMDSDSSPSSSSTYCNLMMCCRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 69 - protein, 631 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSKESRAKKFQRQHMDSDSSPSSSSTYCNLMMCCRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 70 - protein, 635 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSGLFRKESRAKKFQRQHMDSDSSPSSSSTYCNLMMCCRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 71 - protein, 635 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSGLFRKESRAKKFQRQHMDSDSSPSSSSTYCNLMMCCRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 72 - protein, 631 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSKESRAKKFQRQHMDSDSSPSSSSTYCNQMMCCRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 73 - protein, 631 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSKESRAKKFQRQHMDSDSSPSSSSTYCNQMMCCRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 74 - protein, 635 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSGLFRKESRAKKFQRQHMDSDSSPSSSSTYCNQMMCCRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 75 - protein, 635 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSGLFRKESRAKKFQRQHMDSDSSPSSSSTYCNQMMCCRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 76 - protein, 631 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSKESRAKKFQRQHMDSDSSPSSSSTYCNQMMCCRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 77 - protein, 631 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSKESRAKKFQRQHMDSDSSPSSSSTYCNQMMCCRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 78 - protein, 635 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSGLFRKESRAKKFQRQHMDSDSSPSSSSTYCNQMMCCRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 79 - protein, 635 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSGLFRKESRAKKFQRQHMDSDSSPSSSSTYCNQMMCCRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 80 - protein, 631 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSKESRAKKFQRQHMDSDSSPSSSSTYCNLMMRRRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 81 - protein, 631 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSKESRAKKFQRQHMDSDSSPSSSSTYCNLMMRRRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 82 - protein, 635 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSGLFRKESRAKKFQRQHMDSDSSPSSSSTYCNLMMRRRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 83 - protein, 635 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSGLFRKESRAKKFQRQHMDSDSSPSSSSTYCNLMMRRRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 84 - protein, 631 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSKESRAKKFQRQHMDSDSSPSSSSTYCNLMMRRRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 85 - protein, 631 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSKESRAKKFQRQHMDSDSSPSSSSTYCNLMMRRRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 86 - protein, 635 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSGLFRKESRAKKFQRQHMDSDSSPSSSSTYCNLMMRRRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 87 - protein, 635 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSGLFRKESRAKKFQRQHMDSDSSPSSSSTYCNLMMRRRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 88 - protein, 631 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSKESRAKKFQRQHMDSDSSPSSSSTYCNLMMCCRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 89 - protein, 631 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSKESRAKKFQRQHMDSDSSPSSSSTYCNLMMCCRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 90 - protein, 635 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSGLFRKESRAKKFQRQHMDSDSSPSSSSTYCNLMMCCRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 91 - protein, 635 aa, Artificial Sequence
  Humanized anti-5T4 scFv-Fc-Linker (ASTX)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSGLFRKESRAKKFQRQHMDSDSSPSSSSTYCNLMMCCRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 92 - protein, 631 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSKESRAKKFQRQHMDSDSSPSSSSTYCNLMMCCRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 93 - protein, 631 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSKESRAKKFQRQHMDSDSSPSSSSTYCNLMMCCRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 94 - protein, 635 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSGLFRKESRAKKFQRQHMDSDSSPSSSSTYCNLMMCCRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 95 - protein, 635 aa, Artificial Sequence
  Humanized anti-5T4 scFv-Fc-Linker (ASTC)
EVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSGLFRKESRAKKFQRQHMDSDSSPSSSSTYCNLMMCCRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 96 - protein, 257 aa, Artificial Sequence
  ScFv 5T4-specific humanized (human VH3/VK3 frameworks)
EVQLVESGGGLVEPGGSLRLSCKASGYSFTGYYMHWVREAPGKGLEWVSRINPNNGVTLYNQKFKDRFTISRDNSKNTLYLEMNSLRAEDTAVYYCARSTMITNYVMDYWGEGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIK

SEQ ID NO: 97 - protein, 493 aa, Artificial Sequence
  Humanized anti-5T4 scFv-Fc (ASTC)
EVQLVESGGGLVEPGGSLRLSCKASGYSFTGYYMHWVREAPGKGLEWVSRINPNNGVTLYNQKFKDRFTISRDNSKNTLYLEMNSLRAEDTAVYYCARSTMITNYVMDYWGEGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 493 aa, Artificial Sequence
  Humanized anti-5T4 scFv-Fc (ASTX)
EVQLVESGGGLVEPGGSLRLSCKASGYSFTGYYMHWVREAPGKGLEWVSRINPNNGVTLYNQKFKDRFTISRDNSKNTLYLEMNSLRAEDTAVYYCARSTMITNYVMDYWGEGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTXEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 63 aa, Artificial Sequence
  Poly glutamate tail (PolyE)
VHFDASVEDSTGLFREEEEEEASSSSSEEAEEASSSSSAEEEEGASSSEEEASSSSAEEEEEG

SEQ ID NO: 100 - protein, 4 aa, Artificial Sequence
  CatB sequence
GLFR

SEQ ID NO: 101 - protein, 512 aa, Artificial Sequence
  Anti 5T4 scFv Fc HPRN construct sequence
MGWSCIILFLVATATGAHSEVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 650 aa, Artificial Sequence
  Anti 5T4 scFv Fc HPRN construct sequence
MGWSCIILFLVATATGAHSEVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGCSGGPGGGSKESRAKKFQRQHMDSDSSPSSSSTYCNLMMCCRNMTQGRCKPVNTFVHEPLVDVQNVCFQEKVTCKNGQGNCYKSNSSMHITDCRLTNGSRYPNCAYRTSPKERHIIVACGGSPYVPVHFDASVEDST

SEQ ID NO: 103 - protein, 702 aa, Artificial Sequence
  Anti 5T4 scFv Fc HPRN construct sequence tail
MGWSCIILFLVATATGAHSEVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSGLFRKESRAKKFQRQHMDSDSSPSSSSTYCNLMMCCRKMTQGRCKPVNTFVHEPLVDVQNVCFQEKVTCKNGQGNCYKSNSSMHITDCRLTNGSRYPNCAYRTSPKERHIIVACGGSPYVPVHFDASVEDSTEEEEEEASSSSSEEAEEASSSSSAEEEEGASSSEEEASSSSAEEEEEG

SEQ ID NO: 104 - protein, 4 aa, Artificial Sequence
  CatB sequence
GLVR

SEQ ID NO: 105 - protein, 7 aa, Artificial Sequence
  CatB sequence
GLFRFFG

SEQ ID NO: 106 - protein, 7 aa, Artificial Sequence
  CatB sequence
GLVRAFG

SEQ ID NO: 107 - protein, 7 aa, Artificial Sequence
  CatB sequence
GLFRAFG

SEQ ID NO: 108 - protein, 7 aa, Artificial Sequence
  CatB sequence
LLVRFFG

SEQ ID NO: 109 - DNA, 2124 bp, Artificial Sequence
  CHO modified polynucleotide sequence encoding an immunofusion molecule
atgggctggtcctgcatcatcctgtttctggtggctaccgccacaggcgcgcactctgaggtgcagctggtggaatccggcggaggactggtgcagcctggcggctccctgagactgtcctgcaaggcctccggctactccttcaccggctactacatgcactgggtccgacaggctccaggcaagggcctggaatgggtgtcccggatcaaccccaacaacggcgtgaccctgtacaaccagaagttcaaggaccggttcaccatctcccgggacaactccaagaacaccctgtacctgcagatgaactccctgcgggccgaggacaccgccgtgtactactgcgcccggtccaccatgatcaccaactacgtgatggactactggggccagggcaccctggtcacagtgtctagcggtggaggcggaagtggagggggaggatctggcggtggaggatccgggggaggcggatctggtgggggaggtagtgggggagggggctccgacattcagatgacccagtccccctccagcctgtccgcctctgtgggcgacagagtgaccatcacatgcaaggccagccagtccgtgtccaacgacgtggcctggtatcagcagaagcccggcaaggcccccaagctgctgatctactacacctcctccagatacgctggcgtgccctccagattctccggctctggctccggcaccgacttcacactgaccatctccagcctgcagcccgaggacttcgccacctactactgccagcaggactacaacagcccccccaccttcggcggaggcaccaagctcgagatcaaggctagcacctgtgaacccaagtcctccgacaagacccacacctgtcccccctgccctgcccctgaactgctgggaggcccctccgtgttcctgttccccccaaagcccaaggacaccctgatgatctcccggacccccgaagtgacctgcgtggtggtggacgtgtcccacgaggaccctgaagtgaagttcaattggtacgtggacggcgtggaagtgcacaacgccaagaccaagcccagagaggaacagtacaactccacctaccgggtggtgtccgtgctgaccgtgctgcaccaggactggctgaacggcaaagagtacaagtgcaaggtctccaacaaggccctgcctgctcccatcgaaaagaccatctccaaggccaagggccagccccgcgagcctcaggtgtacacactgccccctagccgggacgagctgaccaagaaccaggtgtccctgacctgcctggtcaagggcttctacccctccgatatcgccgtggaatgggagtccaacggccagccagagaacaactacaagaccaccccccctgtgctggactccgacggctcattcttcctgtactccaagctgaccgtggacaagtcccggtggcagcagggcaacgtgttctcctgctccgtgatgcacgaggccctgcacaaccactacacccagaagtccctgtccctgagccccggcaagggctccagtggtggaccaggcggaggctccggcctgttcagaaaagagtcccgggccaagaagttccagcggcagcacatggacagcgactccagcccctccagctcctccacctactgcaacctgatgatgtgctgccggaaaatgacccagggccggtgcaagcccgtgaacaccttcgtgcacgagcccctggtggatgtgcagaacgtgtgttttcaagaaaaagtcacttgcaagaacggccagggcaactgctacaagtccaactcctccatgcacatcaccgactgccggctgaccaacggctccagataccccaactgcgcctaccggacctcccccaaagaacggcacatcatcgtggcctgcggcggctccccttacgtgccagtgcacttcgacgcctccgtggaagattccaccggcctgttcagagaggaggaggaggaggaggcctcttcatccagtagcgaagaagcggaggaggctagctcctcctctagcgccgaggaggaggagggcgcttctagcagcgaggaggaggccagtagctcttccgccgaagaggaagaggaaggatgatga

SEQ ID NO: 110 - DNA, 1542 bp, Artificial Sequence
  CHO modified polynucleotide sequence encoding an immunofusion molecule
atgggctggtcctgcatcatcctgtttctggtggctaccgccacaggcgcgcactctgaggtgcagctggtggaatccggcggaggactggtgcagcctggcggctccctgagactgtcctgcaaggcctccggctactccttcaccggctactacatgcactgggtccgacaggctccaggcaagggcctggaatgggtgtcccggatcaaccccaacaacggcgtgaccctgtacaaccagaagttcaaggaccggttcaccatctcccgggacaactccaagaacaccctgtacctgcagatgaactccctgcgggccgaggacaccgccgtgtactactgcgcccggtccaccatgatcaccaactacgtgatggactactggggccagggcaccctggtcacagtgtctagcggtggaggcggaagtggagggggaggatctggcggtggaggatccgggggaggcggatctggtgggggaggtagtgggggagggggctccgacattcagatgacccagtccccctccagcctgtccgcctctgtgggcgacagagtgaccatcacatgcaaggccagccagtccgtgtccaacgacgtggcctggtatcagcagaagcccggcaaggcccccaagctgctgatctactacacctcctccagatacgctggcgtgccctccagattctccggctctggctccggcaccgacttcacactgaccatctccagcctgcagcccgaggacttcgccacctactactgccagcaggactacaacagcccccccaccttcggcggaggcaccaagctcgagatcaaggctagcacatgcgagcccaagtcctccgacaagacccacacctgtcccccctgccctgcccctgaactgctgggcggaccctccgtgttcctgttccccccaaagcccaaggacaccctgatgatctcccggacccccgaagtgacctgcgtggtggtggacgtgtcccacgaggaccctgaagtgaagttcaattggtacgtggacggcgtggaagtgcacaacgccaagaccaagcccagagaggaacagtacaactccacctaccgggtggtgtccgtgctgaccgtgctgcaccaggactggctgaacggcaaagagtacaagtgcaaggtctccaacaaggccctgcctgcccccatcgaaaagaccatcagcaaggccaagggccagccccgcgagcctcaggtgtacaccctgcctcccagccgggacgagctgaccaagaaccaggtgtccctgacctgcctggtcaaaggcttctacccctccgatatcgccgtggaatgggagtccaacggccagcccgagaacaactacaagaccaccccccctgtgctggactccgacggctcattcttcctgtactccaagctgaccgtggacaagtcccggtggcagcagggcaacgtgttctcctgctccgtgatgcacgaggccctgcacaaccactacacccagaagtccctgtccctgagccccggcaagtgatga

SEQ ID NO: 111 - protein, 1956 aa, Artificial Sequence
  CHO modified polynucleotide sequence encoding an immunofusion molecule
ATGGGCTGGTCCTGCATCATCCTGTTTCTGGTGGCTACCGCCACAGGCGCGCACTCTGAGGTGCAGCTGGTGGAATCCGGCGGAGGACTGGTGCAGCCTGGCGGCTCCCTGAGACTGTCCTGCAAGGCCTCCGGCTACTCCTTCACCGGCTACTACATGCACTGGGTCCGACAGGCTCCAGGCAAGGGCCTGGAATGGGTGTCCCGGATCAACCCCAACAACGGCGTGACCCTGTACAACCAGAAGTTCAAGGACCGGTTCACCATCTCCCGGGACAACTCCAAGAACACCCTGTACCTGCAGATGAACTCCCTGCGGGCCGAGGACACCGCCGTGTACTACTGCGCCCGGTCCACCATGATCACCAACTACGTGATGGACTACTGGGGCCAGGGCACCCTGGTCACAGTGTCTAGCGGTGGAGGCGGAAGTGGAGGGGGAGGATCTGGCGGTGGAGGATCCGGGGGAGGCGGATCTGGTGGGGGAGGTAGTGGGGGAGGGGGCTCCGACATTCAGATGACCCAGTCCCCCTCCAGCCTGTCCGCCTCTGTGGGCGACAGAGTGACCATCACATGCAAGGCCAGCCAGTCCGTGTCCAACGACGTGGCCTGGTATCAGCAGAAGCCCGGCAAGGCCCCCAAGCTGCTGATCTACTACACCTCCTCCAGATACGCTGGCGTGCCCTCCAGATTCTCCGGCTCTGGCTCCGGCACCGACTTCACACTGACCATCTCCAGCCTGCAGCCCGAGGACTTCGCCACCTACTACTGCCAGCAGGACTACAACAGCCCCCCCACCTTCGGCGGAGGCACCAAGCTCGAGATCAAGGCTAGCACCTGTGAACCCAAGTCCTCCGACAAGACCCACACCTGTCCCCCCTGCCCTGCCCCTGAACTGCTGGGAGGCCCCTCCGTGTTCCTGTTCCCCCCAAAGCCCAAGGACACCCTGATGATCTCCCGGACCCCCGAAGTGACCTGCGTGGTGGTGGACGTGTCCCACGAGGACCCTGAAGTGAAGTTCAATTGGTACGTGGACGGCGTGGAAGTGCACAACGCCAAGACCAAGCCCAGAGAGGAACAGTACAACTCCACCTACCGGGTGGTGTCCGTGCTGACCGTGCTGCACCAGGACTGGCTGAACGGCAAAGAGTACAAGTGCAAGGTCTCCAACAAGGCCCTGCCTGCTCCCATCGAAAAGACCATCTCCAAGGCCAAGGGCCAGCCCCGCGAGCCTCAGGTGTACACACTGCCCCCTAGCCGGGACGAGCTGACCAAGAACCAGGTGTCCCTGACCTGCCTGGTCAAGGGCTTCTACCCCTCCGATATCGCCGTGGAATGGGAGTCCAACGGCCAGCCCGAGAACAACTACAAGACCACCCCCCCTGTGCTGGACTCCGACGGCTCATTCTTCCTGTACTCCAAGCTGACCGTGGACAAGTCCCGGTGGCAGCAGGGCAACGTGTTCTCCTGCTCCGTGATGCACGAGGCCCTGCACAACCACTACACCCAGAAGTCCCTGTCCCTGAGCCCCGGCAAGGGCTGCAGTGGTGGACCAGGCGGAGGCTCCAAAGAGTCCCGGGCCAAGAAGTTCCAGCGGCAGCACATGGACAGCGACTCCAGCCCCTCCAGCTCCTCCACCTACTGCAACCTGATGATGTGCTGCCGGAACATGACCCAGGGCCGGTGCAAGCCCGTGAACACCTTCGTGCACGAGCCCCTGGTGGATGTGCAGAACGTGTGTTTTCAAGAAAAAGTCACTTGCAAGAACGGCCAGGGCAACTGCTACAAGTCCAACTCCTCCATGCACATCACCGACTGCCGGCTGACCAACGGCTCCAGATACCCCAACTGCGCCTACCGGACCTCCCCCAAAGAACGGCACATCATCGTGGCCTGCGGCGGCTCCCCTTACGTGCCAGTGCACTTCGACGCCTCCGTGGAAGATTCCACCTGATGA

SEQ ID NO: 112 - DNA, 2112 bp, Artificial Sequence
  CHO modified polynucleotide sequence encoding an immunofusion molecule
atgggctggtcctgcatcatcctgtttctggtggctaccgccacaggcgcgcactctgaggtgcagctggtggaatccggcggaggactggtgcagcctggcggctccctgagactgtcctgcaaggcctccggctactccttcaccggctactacatgcactgggtccgacaggctccaggcaagggcctggaatgggtgtcccggatcaaccccaacaacggcgtgaccctgtacaaccagaagttcaaggaccggttcaccatctcccgggacaactccaagaacaccctgtacctgcagatgaactccctgcgggccgaggacaccgccgtgtactactgcgcccggtccaccatgatcaccaactacgtgatggactactggggccagggcaccctggtcacagtgtctagcggtggaggcggaagtggagggggaggatctggcggtggaggatccgggggaggcggatctggtgggggaggtagtgggggagggggctccgacattcagatgacccagtccccctccagcctgtccgcctctgtgggcgacagagtgaccatcacatgcaaggccagccagtccgtgtccaacgacgtggcctggtatcagcagaagcccggcaaggcccccaagctgctgatctactacacctcctccagatacgctggcgtgccctccagattctccggctctggctccggcaccgacttcacactgaccatctccagcctgcagcccgaggacttcgccacctactactgccagcaggactacaacagcccccccaccttcggcggaggcaccaagctcgagatcaaggctagcacctgtgaacccaagtcctccgacaagacccacacctgtcccccctgccctgcccctgaactgctgggaggcccctccgtgttcctgttccccccaaagcccaaggacaccctgatgatctcccggacccccgaagtgacctgcgtggtggtggacgtgtcccacgaggaccctgaagtgaagttcaattggtacgtggacggcgtggaagtgcacaacgccaagaccaagcccagagaggaacagtacaactccacctaccgggtggtgtccgtgctgaccgtgctgcaccaggactggctgaacggcaaagagtacaagtgcaaggtctccaacaaggccctgcctgctcccatcgaaaagaccatctccaaggccaagggccagccccgcgagcctcaggtgtacacactgccccctagccgggacgagctgaccaagaaccaggtgtccctgacctgcctggtcaagggcttctacccctccgatatcgccgtggaatgggagtccaacggccagcccgagaacaactacaagaccaccccccctgtgctggactccgacggctcattcttcctgtactccaagctgaccgtggacaagtcccggtggcagcagggcaacgtgttctcctgctccgtgatgcacgaggccctgcacaaccactacacccagaagtccctgtccctgagccccggcaagggctccagtggtggaccaggcggaggctccggcctgttcagaaaagagtcccgggccaagaagttccagcggcagcacatggacagcgactccagcccctccagctcctccacctactgcaacctgatgatgtgctgccggaaaatgacccagggccggtgcaagcccgtgaacaccttcgtgcacgagcccctggtggatgtgcagaacgtgtgttttcaagaaaaagtcacttgcaagaacggccagggcaactgctacaagtccaactcctccatgcacatcaccgactgccggctgaccaacggctccagataccccaactgcgcctaccggacctcccccaaagaacggcacatcatcgtggcctgcggcggctccccttacgtgccagtgcacttcgacgcctccgtggaagattccaccgaggaggaggaggaggaggcctcttcatccagtagcgaagaagcggaggaggctagctcctcctctagcgccgaggaggaggagggcgcttctagcagcgaggaggaggccagtagctcttccgccgaagaggaagaggaaggatgatga

SEQ ID NO: 113 - protein, 706 aa, Artificial Sequence
  Immunifusion protein encoded by CHO modified polynucleotide sequence encoding an immunofusion molecule (SEQ ID NO109)
MGWSCIILFLVATATGAHSEVQLVESGGGLVQPGGSLRLSCKASGYSFTGYYMHWVRQAPGKGLEWVSRINPNNGVTLYNQKFKDRFTISRDNSKNTLYLQMNSLRAEDTAVYYCARSTMITNYVMDYWGQGTLVTVSSGGGGSGGGGSGGGGSGGGGSGGGGSGGGGSDIQMTQSPSSLSASVGDRVTITCKASQSVSNDVAWYQQKPGKAPKLLIYYTSSRYAGVPSRFSGSGSGTDFTLTISSLQPEDFATYYCQQDYNSPPTFGGGTKLEIKASTCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGPGGGSGLFRKESRAKKFQRQHMDSDSSPSSSSTYCNLMMCCRKMTQGRCKPVNTFVHEPLVDVQNVCFQEKVTCKNGQGNCYKSNSSMHITDCRLTNGSRYPNCAYRTSPKERHIIVACGGSPYVPVHFDASVEDSTGLFREEEEEEASSSSSEEAEEASSSSSAEEEEGASSSEEEASSSSAEEEEEG